(19) United States
(12) Patent Application Publication
(10) Pub. No.: US 2022/0249823 A1
(43) Pub. Date: Aug. 11, 2022

(54) VASCULAR ACCESS DEVICE AND METHOD

(71) Applicant: ALL VASCULAR PTY LIMITED, Greenwich, New South Wales (AU)

(72) Inventors: Scott Ryan MURPHY, (US); Chris KYUNG, (US); Christopher Michael ROGAN, (US); Nyan Ye KHIN, (US); David Michael LANE, (US); Rodney James LANE, (US)

(21) Appl. No.: 17/754,938

(22) PCT Filed: Oct. 16, 2020
(86) PCT No.: PCT/AU2020/051121

§ 371 Date: Apr. 15, 2022

(30) Foreign Application Priority Data
Oct. 16, 2019 (AU) 2019903897

(51) Int. Cl. A61M 39/02; A61M 29/00; A61M 25/09

(52) U.S. Cl. CPC A61M 39/0247; A61M 29/00; A61M 25/09; A61M 2039/0279; A61M 2039/0261; A61M 2039/0291; A61M 2039/0258

(57) ABSTRACT

The present disclosure concerns a vascular access device (1) comprising a vascular access tube (2), such as a cannula, having a distal end region (3) terminating in a tip (4) for insertion into a blood vessel (V) of a patient and at least one lumen (5) for infusing a medicament and/or for introducing one or more catheters there-through into the blood vessel (V). The device (1) further comprises a fixation mechanism (10) that is operable to secure or fix the distal end region (3) of the vascular access tube (2) with respect to the patient to inhibit or prevent withdrawal of the tip (4) from the blood vessel (V) and/or to inhibit or prevent overinsertion of the tip (4) into the blood vessel (V). The tip (4) of the vascular access tube (2) has an opening (6) for communication between the at least one lumen (5) of the access tube (2) and the blood vessel (V) of the patient into which the tip (4) is inserted, and the fixation mechanism (10) is desirably configured to position and fix the opening (6) of the tip (4) at or adjacent a wall (W) of the blood vessel (V) at a point of insertion of the tip (4). The fixation mechanism (10) is preferably configured to secure or fix the vascular access tube such that the access tube extends longitudinally at a predetermined angle (#) with respect to the blood vessel (V) at a point of insertion of the tip (4). The predetermined angle (#) may be within the range of about 20 degrees to about 90 degrees. The disclosure provides a corresponding method of implanting the vascular access device (1) in a patient.

### TECHNICAL FIELD

The present invention relates to a vascular access device that is particularly suited to percutaneous insertion for use in patients requiring multiple vascular access procedures over an extended period of time. This invention also relates to a method of implanting a vascular access device in a patient.

As noted above, the vascular access device and the associated method of this disclosure are especially suited to percutaneous use and it will be convenient to herein describe the vascular access device and associated method in this exemplary context. It will be appreciated, however, that the device and method are not limited to such a use or application, but that they may also be employed in a surgical procedure; for example, where a surgical intervention may be required to reach the blood vessel into which the vascular access device is to be introduced, and introduction of the device is performed in a transvascular manner.

### BACKGROUND ART

Vascular access tubes for infusing a medicament and/or for introducing one or more catheters therethrough into the blood vessel of a patient are known. Some known vascular access tubes for insertion percutaneously into the vasculature are designed to be inserted with an appreciable length of the tube protruding into and aligning with the lumen of the blood vessel. This has the disadvantage, however, that disturbances and restrictions in blood flow through the vessel can be created by the length of tube that resides in the blood vessel, as it occupies a portion of the vessel cross-section equal to the cross-sectional area of the tube itself. The access tube may thus create significant ongoing thrombotic/thrombo-embolic, ischaemic and intimal hyperplasia risks while the access tube is in situ. Further, the risk of colonisation of device surfaces and eventual bloodstream infection resulting from such a colonisation is generally proportional to the blood-contacting surface area of the device.

Some other known vascular access tubes, e.g. for providing arterial access, are configured with peripheral graft material which is designed to be sutured to the tissue of the blood vessel. While such a graft arrangement may allow the access tube to provide communication with the artery without having an appreciable length of the access tube protruding into and possibly obstructing the lumen of the blood vessel, the procedure for introducing and securing such arterial access tubes must be performed via an involved surgical procedure. Specifically, the complexity and labour-intensity of implanting such graft devices surgically, which typically includes surgical cutdown, the trimming graft material to a correct shape or configuration for anastomosis and then anastomosing the graft material to the vessel by suturing, is substantial. As a result, this form of vascular access consumes considerably more time and hospital resources than percutaneous or transvascular insertion.

In view of the above, therefore, it would be desirable to provide a new vascular access device that is suitable for percutaneous deployment or implantation, and that is able to reduce or substantially minimise or avoid unnecessary obstruction of the blood vessel. It would also be desirable to provide a corresponding method of vascular access that is particularly suited, though not limited to, use in percutaneous procedure.

### SUMMARY

In one aspect, the present disclosure provides a vascular access device comprising:

a vascular access tube having a distal end region terminating in a tip to be inserted into a blood vessel of a patient and having at least one lumen, e.g. for infusing a medicament and/or for introducing one or more catheters there-through into the blood vessel, wherein the tip has an opening to provide communication between the at least one lumen of the vascular access tube and the blood vessel; and

a fixation mechanism operable to secure or fix the tip of the vascular access tube within the blood vessel, wherein the fixation mechanism is configured to secure or fix the tip opening at or adjacent a wall of the blood vessel at a location where the tip enters the blood vessel.

In this way, the fixation mechanism is configured to secure or fix the tip of the vascular access tube within the blood vessel at or adjacent the wall of the blood vessel at the point of insertion; that is, with minimal intrusion into the vessel lumen. Minimising intrusion of the access tube into the blood vessel thus reduces flow disturbances and restrictions in the accessed blood vessel, as well as the surface area of foreign device materials exposed to circulating blood. This vascular access device may thus provide significant reduction in ongoing thrombotic/thromboembolic, ischaemic and intimal hyperplasia risks while the device is in-situ compared to typical access tube designs where an appreciable length of the device protrudes into blood vessel and occupies a portion of the vessel cross-section.

In the context of the present invention, it will be appreciated by ordinary skilled persons in the art that the “vascular access tube” may be in the form of a cannula or an introducer sheath. In this regard, as noted above, the access tube may be suitable for infusing material to, and/or aspirating material from, the blood vessel as well as for introducing one or more catheters there-through into the blood vessel. That is, the at least one lumen of the access tube or cannula may be adapted for introducing one or more intravascular devices and/or for the extraction and/or return of bodily fluid.

In a preferred embodiment, the fixation mechanism that is configured to inhibit or prevent withdrawal of the tip from the blood vessel and/or to inhibit or prevent over-insertion of the tip into the blood vessel. To this end, the fixation mechanism is provided, at least in part, at a distal end region of the vascular access tube. As such, the fixation mechanism operates to provide a stable connection between the access tube and the blood vessel. In this way, the fixation mechanism is designed to resist loads applied to the device in use, including unavoidable impulses associated with use, such as blood pressure, pressure conditions from infusion or extraction operations, transmitted forces from use of catheters through the device, and advancement/withdrawal of an occlude or dilator; as well as normal handling and inadvertent knocks or impulses applied to the device in use. In particular, the fixation mechanism preferably operates to inhibit withdrawal and/or to inhibit over-insertion of the tip with respect to the blood vessel due to loads applied in use, including any inadvertent knock or impulse to the device.

In another aspect, therefore, the disclosure provides a vascular access device, comprising:

a vascular access tube with a distal end region terminating in a tip for insertion into a blood vessel of a patient and at least one lumen for infusing medicament and/or for introducing one or more catheters there-through into the blood vessel; and

a fixation mechanism that is operable to secure or fix the distal end region of the vascular access tube with respect to the patient to inhibit or prevent withdrawal of the tip from the blood vessel and/or over-insertion of the tip into the blood vessel.

In this way, the fixation mechanism in the vascular access device can operate to provide a stable connection between the access tube and the blood vessel; i.e. one that inhibits or prevents inadvertent withdrawal of the tip out of the blood vessel and/or one that inhibits or prevents over-insertion of the tip into the blood vessel. Accordingly, the fixation mechanism can be configured to secure or fix the tip of the vascular access tube within the blood vessel positioned at a location adjacent a wall of the blood vessel through which the tip is inserted; that is, with minimal intrusion into the vessel lumen.

In this context, it will be noted that the proximal end of the vascular access tube is typically located extracorporeally (i.e. outside of the patient's body) for use in infusing material to, and/or aspirating material from, the blood vessel as well as for introducing one or more catheter (or other intravascular device) there-through into the blood vessel. The proximal end of the vascular access tube or cannula could thus be connected to a variety of adaptors or other devices for the purposes of infusion, extraction of bodily fluids, or introduction of intravascular devices. With the addition of an occluder element to close and preserve the lumen of the access tube against thrombosis (also the subject of previous patent applications by the present Applicant), the vascular access device of this disclosure may also permit long-term use with improved safety and longevity when compared to conventional introducer sheath designs. The vascular access device of this disclosure further provides the additional possibility of the access tube having a larger lumen suitable for the introduction of multiple catheter-like devices with acceptable patient safety that is not possible with conventional introducer sheaths. In an alternative case, however, it will be appreciated that a proximal end of the vascular access tube or cannula could be in a subcutaneous location where, for example, it could be connected to an infusion pump or reservoir for infusion of a medicament into the blood vessel.

In a preferred embodiment, the tip of the vascular access tube has an opening for providing communication between the lumen(s) of the vascular access tube and the blood vessel of the patient into which the tip is inserted. Further, the fixation mechanism is configured to position and to secure or fix the opening of the tip at or adjacent a wall of the blood vessel at a point of insertion of the tip. In this way, the tip of the access tube and its distal opening are at or adjacent the wall of the blood vessel through which the tip is inserted, such that the tip does not substantially project into the blood vessel.

In a preferred embodiment, the fixation mechanism comprises an intravascular part that is configured to engage with an inner surface of the wall of the blood vessel through which the tip is inserted to inhibit or prevent withdrawal of the tip of the vascular access tube from the blood vessel. Preferably, the intravascular part is configured to engage the inner surface of the blood vessel wall at a periphery of a breach in the wall through which the tip of the access tube is inserted. In this regard, the intravascular part of the fixation mechanism may comprise at least one member that is fixed relative to the access tube and configured to engage with or bear against an inner surface of the wall of the blood vessel when the tip of the access tube is inserted. For example, the at least one member could be a fixed or static flange, flap, or arm member projecting from the tip of the access tube. After introduction of the tip of the access tube or cannula into the vessel, elastic recovery of the dilated tissue causes the vessel wall to contract around a periphery of the cannula, and within the extent of the fixed flange, flap, or arm member.

Preferably, the intravascular part of the fixation mechanism comprises at least one member that is movable between a retracted or non-deployed position, for insertion of the tip of the access tube into the blood vessel, and an extended or deployed position for engaging the inner surface of the wall of the blood vessel after insertion to inhibit or prevent withdrawal of the tip. To this end, the at least one member of the intravascular part is typically configured to project laterally or radially outwardly from the access tube in its extended or deployed position. In a preferred embodiment, the intravascular part of the fixation mechanism comprises a plurality of members that are movable between a retracted or non-deployed position, i.e. for insertion of the tip of the access tube into the blood vessel, and an extended or deployed position for engaging the inner surface of the wall of the blood vessel after insertion to inhibit or prevent withdrawal of the tip. The at least one member of the intravascular part of the fixation mechanism may optionally have or support a flexible membrane or web-like covering.

In an embodiment, the at least one member of the intravascular part comprises a flange, a flap, or an arm member for engaging and bearing against an inner surface or inner side of the wall of the blood vessel. In an embodiment, the at least one member of the intravascular part comprises a barb or prong member configured to engage and bear against the inner side of the wall of the blood vessel, and optionally also to pierce the wall of the blood vessel. In this regard, it will be noted that active fixation elements of this type, such as barbs or prongs, may pierce through the full thickness of the vessel wall, or they may only cut into a partial wall thickness to lodge in the tissue. Such a barb or prong may optionally be combined with any one or more of a flange member, a flap member, or an arm member in the intravascular part of the fixation mechanism.

In a preferred embodiment, the at least one member of the intravascular part of the fixation mechanism may comprise a framework, e.g. in the manner of a stent- or mesh-like structure. In this regard, the at least one member may be comprised of multiple fine interconnected elements, optionally of metal (wire), such as stainless steel or Nitinol, or of a bio-compatible polymer plastic material. This at least one member may optionally include a flexible membrane or web overlying or covering the framework.

In a preferred embodiment, the fixation mechanism comprises an extravascular part configured to engage with tissue of the patient outside of the blood vessel. In this regard, the extravascular part may be configured to inhibit or prevent over-insertion of the tip of the vascular access tube into the blood vessel. The extravascular part may, for example, be configured to engage with either an outer surface of the wall of the blood vessel through which the tip is inserted or with tissue immediately adjacent the vessel, that may possibly encase the vessel. That is, the extravascular part may be configured to engage the outer surface of the blood vessel wall or adjoining tissue at or around a periphery of the breach in the wall through which the tip of the access tube is inserted.

In a particularly preferred embodiment, the extravascular part of the fixation mechanism is configured to cooperate with the intravascular part to capture or sandwich the wall of the blood vessel there-between. In this regard, both the intravascular part and the extravascular part may engage the wall of the blood vessel at or around a periphery of the breach in the wall through which the tip of the access tube is inserted.

In a preferred embodiment, the extravascular part of the fixation mechanism includes at least one member that is movable between a retracted or non-deployed position, which it will assume during insertion of the tip of the access tube into the blood vessel, and an extended or deployed position to inhibit or prevent an over-insertion of the tip. In this regard, the at least one member of the extravascular part is preferably designed to engage an outer surface or outer side of the wall of the blood vessel in a deployed position. For example, the at least one member of the extravascular part may comprise a flange member, a flap member, or an arm member for engaging or bearing against an outer surface of the wall of the blood vessel. In another example, the at least one member of the extravascular part may include a collar or clamp movable along a periphery of the access tube for engaging or bearing against an outer surface of the wall of the blood vessel or tissue adjoining the blood vessel that may, for example, encase the vessel. In a further example, the at least one member of the extravascular part may comprise one or more barb or prong for projecting into and engaging subcutaneous tissue of the patient in its deployed position. In yet another example, the at least one member of the extravascular part may include a collar or clip movable along a periphery of the access tube for engaging or bearing against the skin of the patient where the access tube or cannula emerges percutaneously, to be fixed in that position. The at least one member of the extravascular part of the fixation mechanism may optionally have or support a flexible membrane or web-like covering.

In a preferred embodiment, the extravascular part of the fixation mechanism includes a plurality of members that are movable between a retracted or non-deployed position and an extended or deployed position for engaging an outer surface or outer side of the wall of the blood vessel after insertion to inhibit or prevent over-insertion of the tip. The above examples of at least one member of the extravascular part of the fixation mechanism may be provided in any multiples and/or in any suitable combination with one another.

In a preferred embodiment, the at least one member of the extravascular part of the fixation mechanism may comprise a framework, such as in the manner of a stent- or mesh-like structure. To this end, the at least one member may be comprised of multiple fine interconnected elements, optionally of metal, such as stainless steel or nitinol wire, or of a bio-compatible polymer plastic material. This at least one member may optionally include a membrane overlying or covering the framework.

Different insertion techniques are contemplated for insertion or implantation of the vascular access device of the invention into a patient's blood vessel. One technique, for example, would be to insert the tip of the access tube through the wall of the blood vessel over a guidewire with the aid of a dilator in a variation of the Seldinger technique. In this regard, the dilator can be a tapered element which is threaded on the guidewire to provide gradual, atraumatic dilation or expansion of the breach in the vessel wall for insertion of the tip of the access tube with minimal force or trauma to the vessel. The dilator element may be combined with the cannula in an interference fit at the interface between the leading edge of the cannula and the underlying surface of the dilator for an atraumatic transition. The dilator element may be removed through that lumen after the tip of the access tube has been inserted through the vessel wall. Another technique may involve the insertion or implantation of the vascular access device of the invention using a percutaneous delivery sheath, which is inserted percutaneously at the intended site of implantation.

In a preferred embodiment, therefore, the vascular access device includes a delivery sheath for assisting percutaneous insertion and positioning of the vascular access tube relative to the blood vessel. The delivery sheath typically accommodates or surrounds the vascular access tube and has a distal tip to be placed in the blood vessel (e.g. in a variation of the Seldinger technique discussed above) and via which the tip of the vascular access tube is introduced through the wall of the blood vessel.

In a preferred embodiment, the at least one member (and optionally multiple members) of the intravascular part of the fixation mechanism are adapted to be actively, and preferably repeatedly, operated or moved between the retracted or non-deployed position and the extended or deployed position. Also, in a preferred embodiment, the at least one member (and optionally multiple members) of the extravascular part of the fixation mechanism are adapted to be actively, and preferably repeatedly, operated or moved between the retracted or non-deployed position and the extended or deployed position.

In a preferred embodiment, the fixation mechanism comprises at least one activation member (optionally multiple activation members) for activating or operating the intravascular part and/or the extravascular part of the fixation mechanism during insertion of the tip of the access tube into the blood vessel. In particular, the activation member(s) is/are adapted to move or operate the member(s) of the intravascular and/or extravascular parts of the fixation mechanism between their respective retracted or non-deployed positions and their respective extended or deployed positions. This way, each activation member may be operably associated or connected with the distal end region of the vascular access tube. The or each activation member is configured for operation by a user at a proximal end region of the access tube to activate or operate the fixation mechanism during insertion of the tip of the access tube into the blood vessel.

In a particularly preferred example, the activation member comprises a sheath or sleeve, especially the delivery sheath noted above, that covers the distal end region of the vascular access tube during insertion of the tip into the blood vessel. The sheath or sleeve is configured to be withdrawn or retracted from the distal end region, whereby withdrawal or retraction of the sheath or sleeve operates to cause movement of the at least one member of the intravascular part and/or the at least one member of the extra-vascular part of the fixation mechanism from its respective retracted or non-deployed position to its extended or deployed position. In particular, the withdrawal or retraction of the sheath or sleeve may release or free the member(s) of the intravascular and/or extravascular parts of the fixation mechanism for movement from a respective retracted or non-deployed position to the extended or deployed position. In this context, each member of the intravascular and/or extravascular parts of the fixation mechanism may be resiliently biased to move from its respective retracted or non-deployed position to its extended or deployed position automatically upon withdrawal or retraction of the sheath or sleeve. In an alternative arrangement, however, the act of withdrawing the sheath or sleeve may actively urge the member(s) of the intravascular and/or extravascular parts to its/their respective extended or deployed position(s).

In a preferred embodiment, instead of or additional to an outer sheath or sleeve, it will be noted that the activation member could be provided in the form of a flexible member, such as a cord or line, to be drawn (i.e. pulled under tension) by an operator at a proximal end region of the vascular access device, with the cord or line preferably extending from the distal end region. In this way, the activation impulse can be imparted or transmitted via the tension force in the flexible member to the distal end to activate the member(s) of the intravascular part and/or the extra-vascular part of the fixation mechanism.

In a further embodiment, the activation member could be provided in the form of a rigid member, such as a rod or (partial) tube, to be pressed or moved by an operator at the proximal end region of the vascular access device, with the rigid member (e.g. the rod or tube) connected or extending to the distal end region to transmit or impart the impulse applied by the user. In this way, the activation member could, for example, be provided as an internal sheath or sleeve (e.g. such as a liner) which is configured to be inserted and/or retracted within the lumen of the access tube.

In a preferred embodiment, the fixation mechanism is configured to secure or fix the vascular access tube with respect to the patient such that it extends longitudinally at a predetermined angle with respect to an extent of the blood vessel at a point of insertion of the tip into the blood vessel. In this regard, the predetermined angle may be selected anywhere in the range of 0 to 90 degrees. In one particular preferred example, the predetermined angle of the access tube to the blood vessel is about 90 degrees. Alternatively, the predetermined angle is typically within the range of about 20 degrees to about 70 degrees. Examples of the preferred predetermined angle include: about 30 degrees; about 45 degrees; and about 60 degrees. Thus, the intravascular part and/or the extravascular part of the fixation mechanism are adapted to interact with the wall of the blood vessel and/or to cooperate with one another to fix or secure the access tube such that it extends longitudinally at the predetermined angle with respect to the blood vessel at the point of insertion of the tip into the blood vessel.

In a preferred embodiment, the vascular access device includes a protector or guard member configured to prevent a member or element of the fixation mechanism, and especially of the intravascular part of the fixation mechanism, from inadvertently making contact or interacting with the blood vessel or with a haemostasis valve of a tear-away introducer sheath, during the insertion of the access tube or cannula into the blood vessel. To this end, the guard member preferably includes a recess adjacent to its distal end to accommodate members or elements of the fixation mechanism.

In a particularly preferred embodiment, the protector or guard member includes a chamber or cavity configured to accommodate, encompass, or substantially house the said members or elements of the fixation mechanism. For example, side walls of the protector or guard member may define or at least partially enclose a cavity or chamber for accommodating members or elements of the fixation mechanism. The protector or guard member is thus designed to protect both the patient as well as other equipment associated with the procedure from unwanted trauma or damage caused by inadvertent contact with elements of the fixation mechanism during insertion of the vascular access device; i.e. before the tip of the access tube or cannula has reached a position at which the fixation mechanism is ready to be deployed; and/or during extraction of the vascular access device. Examples of susceptible areas to be protected include: an opposite wall of the implanted vessel; the breach/puncture in the vessel wall through which the device is inserted or extracted (e.g. percutaneously or transvascularly); extravascular tissue (e.g. skin or subcutaneous tissues) along the path of insertion/extraction (i.e. between the skin entry point and the blood vessel); and a haemostasis valve of any introducer sheath used to facilitate deployment of the device. To this end, the protector or guard member may provide a blunt, atraumatic distal end-stop for the device during insertion or introduction of the device into a blood vessel. More specifically, the protector or guard member preferably has a distal end configured to be flat or rounded and optionally formed from a relatively soft, flexible material designed to avoid inflicting any trauma on tissue. Thus, the protector or guard member preferably has atraumatic geometries, such as smooth surfaces and no sharp corners, edges, or burrs, at locations that could contact or interact with tissue of the patient, such as the blood vessel wall or haemostasis valve.

In yet another aspect, the present disclosure provides a vascular access system comprising:

a vascular access device according to any one of the embodiments described above; and

a dilator for gradually widening a breach or an opening formed in a wall of a blood vessel, wherein the dilator is adapted to cooperate with the access tube to guide and/or introduce the tip of the distal end region of the access tube through the breach or opening in the wall of the blood vessel.

In a preferred embodiment, the tip of the access tube is configured to provide a smooth or gradual, preferably tapered, transition to an outer periphery of the dilator. In this way, a substantially atraumatic and/or gradual insertion of the tip of the access tube though the expanded or dilated breach in the wall of the vessel can be achieved.

In a preferred embodiment, the dilator is sized and/or adapted to be withdrawn or removed from the patient through the lumen of the access tube. That is, after the tip of the access tube has been successfully inserted into the blood vessel, and the fixation mechanism optionally at least partly deployed, the dilator may be withdrawn or removed in the proximal direction through the lumen of the access tube.

In a preferred embodiment, the dilator includes a recess or chamber configured to house or accommodate one or more members or parts of the fixation mechanism of the access device. In this way, the dilator may be configured to prevent such members or parts of the fixation mechanism (and especially an intravascular part of the fixation mechanism) from inadvertently making contact with and potentially causing damage to patient tissues, like an opposite wall of the blood vessel, or the haemostasis valve of a tear-away introducer sheath, during the insertion of the vascular access device into the blood vessel and before the tip of the access tube or cannula has reached a position at which the fixation mechanism is ready for deployment. Accordingly, the dilator in this embodiment may form a protector or guard member of the type described above.

In a preferred embodiment, the vascular access system includes a guidewire for guiding a path of the tip of the distal end region of the vascular access tube through a breach or an opening formed in a wall of a blood vessel tip of the access tube. In this regard, dilator may include a channel, e.g. centrally or axially, for accommodating the guidewire. The guidewire may thus guide insertion or introduction of the dilator through the breach or opening in the vessel wall, which in turn guides insertion or introduction of the tip of the access tube.

In a further aspect, the present disclosure provides a method of implanting a vascular access device into a patient, the method comprising steps of:

inserting a vascular access tube into a patient, the access tube having a tip that is introduced through a wall of a blood vessel of the patient and at least one lumen for introducing one or more catheters therethrough into the blood vessel; and

activating a fixation mechanism, at least part of which is provided at a distal end region of the vascular access tube, e.g. at or in the vicinity of the tip, to secure or fix the distal end region of the access tube with respect to the patient, whereby the fixation mechanism secures or fixes the tip of the access tube at or adjacent the wall of the blood vessel at a point of entry of the tip through the wall.

In a preferred embodiment, the step of inserting the vascular access tube into the patient, which is preferably performed percutaneously, comprises introducing the tip of the access tube through the wall of the blood vessel over a guidewire with the aid of a dilator. This may, for example, comprise a variation of the Seldinger technique. The dilator may comprise a tapered element which is threaded on the guidewire to provide a gradual, atraumatic dilation or expansion of the breach in the vessel wall for insertion of the tip of the access tube with minimal force or trauma to the vessel. The guidewire-to-dilator transition and the dilator-to-access tube (cannula) transition are usually designed for atraumatic transition. It will be noted that, although the step of inserting the vascular access tube into the patient is preferably performed percutaneously, i.e. non-surgically through the skin, it is also conceivable that an initial surgical procedure could be needed to expose the vessel prior to the step of inserting the vascular access tube in a trans-vascular manner. This may, for example, depend on a location of the blood vessel into which the vascular access device is to be inserted—e.g. the vessel may not be readily accessible percutaneously.

In another preferred embodiment, the step of inserting the vascular access tube into the patient comprises introducing the vascular access tube via a delivery sheath, which may be inserted percutaneously at the intended site of implantation. The vascular access tube is introduced through an interior of the sheath to the site for inserting the tip of the vascular access tube through the breach in the wall of the blood vessel. It will be noted that the sheath may optionally also be inserted via preliminary surgical cutdown followed by trans-vascular introduction in a variation of the Seldinger technique.

In a preferred embodiment, the fixation mechanism acts to inhibit or prevent an inadvertent withdrawal of the tip from the blood vessel.

In a preferred embodiment, the fixation mechanism acts to inhibit or prevent an over-insertion of the tip into the blood vessel.

In a preferred embodiment, activating the fixation mechanism comprises moving at least one member of an intravascular part of the fixation mechanism from a retracted or non-deployed position to an extended or deployed position to engage with an inner surface of the wall of the blood vessel through which the tip has been inserted.

In a preferred embodiment, activating the fixation mechanism comprises moving at least one member of an extravascular part of the fixation mechanism from a retracted or non-deployed position to an extended or deployed position to engage with an outer surface of the wall of the blood vessel through which the tip has been inserted, thereby to inhibit or prevent over-insertion of the tip of the access tube into the blood vessel.

In a preferred embodiment, activating the fixation mechanism comprises moving at least one member of an extravascular part of the fixation mechanism from a retracted or non-deployed position to an extended or deployed position to project into and engage in subcutaneous tissue of the patient to inhibit or prevent over-insertion of the tip of the access tube into the blood vessel.

In a preferred embodiment, the tip of the vascular access tube has at least one opening which provides communication between the at least one lumen of the vascular access tube and the blood vessel of the patient into which the tip is inserted, whereby the fixation mechanism operates to position and to secure or fix the opening of the tip at or adjacent the wall of the blood vessel at a location where the tip is inserted.

In a preferred embodiment, activating the fixation mechanism operates to fix or secure the vascular access tube extending longitudinally at a predetermined angle with respect to an extent of the blood vessel at a point of insertion of the access tube. As noted above, the predetermined angle is preferably in the range of about 20 degrees to about 90 degrees, more preferably in the range of about 40 degrees to 90 degrees.

In a preferred embodiment, the method comprises a step of releasing one or more members or parts of the fixation mechanism from a protector or guard member at a tip of the vascular access tube after the step of inserting the vascular access tube into the patient and before or during the step of activating the fixation mechanism.

In yet another aspect, the disclosure provides a method of explanting a vascular access device from a patient, preferably percutaneously, the vascular access device having a vascular access tube with a tip inserted through a wall of a blood vessel of the patient and defining at least one lumen for introducing one or more catheters therethrough into the blood vessel, the method comprising:

deactivating a fixation mechanism at a distal end region of the vascular access tube, e.g. adjacent to or in the vicinity of the tip, to release the distal end region of the access tube with respect to the patient;

withdrawing the vascular access tube from the patient, e.g. percutaneously.

In a preferred embodiment, the step of deactivating the fixation mechanism comprises moving at least one member of an intravascular part and/or an extravascular part of the fixation mechanism from an extended or deployed position to a retracted or non-deployed position. In this regard, moving the member(s) of the intravascular and/or extravascular part may comprise releasing or (re-)collapsing the member(s), which may in turn involve its/their elastic and/or plastic deformation. That is, the various examples or embodiments of the fixation mechanism described above in the context of activating the fixation mechanism to secure or fix the tip of the access tube at or adjacent the wall of the blood vessel are preferably reversible for moving member(s) of the intravascular part and/or the extravascular part for deactivating the fixation mechanism to release the tip of the access tube from the wall of the blood vessel. The capacity for percutaneous explanation without need for open vascular surgery constitutes a significant advantage in terms of economisation of resources, a reduction of complication rates and improved patient outcomes and patient comfort. Indeed, these advantages attributable to the use of the fixation mechanism apply even when use of the device involves an initial surgical procedure to expose the blood vessel. That is, after an initial surgical step to expose the site, deactivation of the fixation mechanism and transvascular withdrawal of the device should still require significantly less physical and cognitive effort when compared with a full surgical, graft-based device.

In a preferred embodiment, the method of explanting the device includes a step of housing or accommodating one or more members or parts of the fixation mechanism in a protector or guard member at a tip of the vascular access tube after the step of deactivating the fixation mechanism and before removing the vascular access tube from the patient.

In a preferred embodiment, the method further comprises sealing or closing the breach in the wall of the blood vessel. This may, for example, comprise applying any of a range of vascular closure devices or applying a sealant to the blood vessel to promote closure of the breach in the wall of the blood vessel during and/or following the step of withdrawing the vascular access tube from the patient. Surgical closure of the exit site and any other surgical wound may then be necessary.

### BRIEF DESCRIPTION OF THE DRAWINGS

For a more complete understanding of the disclosure and advantages thereof, exemplary embodiments are explained in more detail in the following description with reference to the accompanying drawing figures, in which like reference signs designate like parts and in which:

FIG. 1 is a schematic side view of a known vascular access tube shown in an inserted state;

FIG. 2 is a schematic side view of a vascular access device illustrating a basic concept of the invention;

FIG. 3a is a schematic side view of a vascular access device according to a first embodiment of the invention during insertion into the blood vessel;

FIG. 3b is a schematic side view of the vascular access device of FIG. 3a showing the intravascular part of the fixation mechanism in a deployed position;

FIG. 3c is a schematic side view of the vascular access device of FIG. 3a showing the extravascular part of the fixation mechanism in a deployed position;

FIG. 4a is a schematic side view of a vascular access device according to a second embodiment during percutaneous insertion into the blood vessel;

FIG. 4b is a schematic side view of the vascular access device of FIG. 4a showing the intravascular part of the fixation mechanism in a deployed position;

FIG. 4c is a schematic side view of the vascular access device of FIG. 4a showing the extravascular part of the fixation mechanism in a deployed position;

FIG. 5 is a schematic side view of a vascular access device according to a third embodiment shown inserted into a blood vessel with the fixation mechanism activated;

FIG. 6a is a schematic side view of the extravascular part of the fixation mechanism of the vascular access device of FIG. 5 in a non-deployed position;

FIG. 6b is a schematic side view of the extravascular part of the fixation mechanism of the vascular access device of FIG. 5 in a deployed position;

FIG. 7a is a schematic side view of the extravascular part of a fixation mechanism of a fourth embodiment of a vascular access device similar to FIG. 5 shown in a non-deployed position;

FIG. 7b is a schematic side view of the extravascular part of the fixation mechanism in FIG. 7a shown in a deployed position;

FIG. 8a is a schematic side view of a vascular access device according to a fifth embodiment during percutaneous insertion into the blood vessel;

FIG. 8b is a schematic side view of the vascular access device of FIG. 8a showing the intravascular part of the fixation mechanism during deployment;

FIG. 8c is a schematic side view of the vascular access device of FIG. 8a showing the intravascular part of the fixation mechanism deployed;

FIG. 8d is a schematic side view of the vascular access device of FIG. 8a showing the extravascular part of the fixation mechanism deployed;

FIG. 9 is a schematic side view of a vascular access device according to a sixth embodiment inserted in a blood vessel with fixation mechanism activated;

FIG. 10a is a schematic side view of a vascular access device according to a seventh embodiment during percutaneous insertion into the blood vessel;

FIG. 10b is a schematic side view of the vascular access device of FIG. 10a during the deployment of the intravascular part of the fixation mechanism;

FIG. 10c is a schematic side view of the vascular access device of FIG. 10a still during deployment of the intravascular part of the fixation mechanism;

FIG. 10d is a schematic side view of the access device of FIG. 10a with the intravascular part of the fixation mechanism fully deployed;

FIG. 11a is a schematic side view of a vascular access device according to an eighth embodiment with the intravascular part of the fixation mechanism in a deployed position;

FIG. 11b is a schematic side view of the vascular access device of FIG. 11a showing the extravascular part of the fixation mechanism in a deployed position;

FIG. 12a is a schematic side view of a vascular access device according to a ninth embodiment during percutaneous insertion into the blood vessel;

FIG. 12b is a schematic side view of the vascular access device of FIG. 12a showing the intravascular part of the fixation mechanism during deployment;

FIG. 12c is a schematic side view of the vascular access device of FIG. 12a showing the intravascular part of the fixation mechanism in a deployed position;

FIG. 13a is a schematic side view of a vascular access device according to a tenth embodiment during percutaneous insertion into the blood vessel;

FIG. 13b is a schematic side view of the vascular access device of FIG. 13a showing the intravascular part of the fixation mechanism during deployment;

FIG. 13c is a schematic side view of the vascular access device of FIG. 13a showing the intravascular part of the fixation mechanism in a deployed position;

FIG. 14 is a flow diagram which schematically represents a method according to any of a variety of embodiments of the invention;

FIG. 15a is a schematic side view of an embodiment of a vascular access device after percutaneous insertion into the blood vessel;

FIGS. 15b to 15e are schematic ends views of the vascular access device of FIG. 15a that show various embodiments of the flange member of the intravascular part of the fixation mechanism;

FIG. 16a is a schematic side view of a vascular access device according to an eleventh embodiment during percutaneous insertion into the blood vessel;

FIG. 16b is a schematic end view of the vascular access device of FIG. 16a showing the intravascular part of the fixation mechanism before deployment;

FIG. 16c is a schematic side view of the vascular access device of FIG. 16a showing the intravascular part of the fixation mechanism in a deployed position;

FIG. 16d is a schematic end view of the vascular access device of FIG. 16a showing the intravascular part of the fixation mechanism in a deployed position;

FIG. 17a is a schematic side view of a vascular access device according to a twelfth embodiment during percutaneous insertion into the blood vessel;

FIG. 17b is a schematic end view of the vascular access device of FIG. 17a showing the intravascular part of the fixation mechanism before deployment;

FIG. 17c is a schematic side view of the vascular access device of FIG. 17a showing the intravascular part of the fixation mechanism in a deployed position;

FIG. 17d is a schematic end view of the vascular access device of FIG. 17a showing the intravascular part of the fixation mechanism in a deployed position;

FIG. 18a is a schematic side view of a vascular access device according to a thirteenth embodiment during percutaneous insertion into the blood vessel;

FIG. 18b is a schematic side view of the vascular access device of FIG. 18a showing the intravascular part of the fixation mechanism during deployment;

FIG. 18c is a schematic side view of the vascular access device of FIG. 18a showing the intravascular part of the fixation mechanism during deployment;

FIG. 18d is a schematic side view of the vascular access device of FIG. 18a showing the intravascular part of the fixation mechanism in a deployed position;

FIG. 19a is a schematic side view of a vascular access device according to a fourteenth embodiment upon insertion into the blood vessel;

FIG. 19b is a schematic side view of the vascular access device of FIG. 19a showing the intravascular part of the fixation mechanism during deployment;

FIG. 19c is a schematic side view of the vascular access device of FIG. 19a showing the intravascular part of the fixation mechanism in a deployed position;

FIG. 20a is a schematic side view of a vascular access device according to a fourteenth embodiment upon insertion into the blood vessel;

FIG. 20b is a schematic side view of the vascular access device of FIG. 20a showing the intravascular part of the fixation mechanism during deployment;

FIG. 20c is a schematic side view of the vascular access device of FIG. 20a showing the intravascular part of the fixation mechanism in a deployed position;

FIG. 21a is a schematic side view of a vascular access device according to a fifteenth embodiment after percutaneous insertion into the blood vessel;

FIG. 21b is a schematic side view of the vascular access device of FIG. 21a showing the extravascular part of the fixation mechanism in a deployed position;

FIG. 22a is a schematic side view of a vascular access device according to a sixteenth embodiment after percutaneous insertion into the blood vessel;

FIG. 22b is a schematic side view of the vascular access device of FIG. 22a showing the extravascular part of the fixation mechanism in a deployed position;

FIG. 23a is a schematic side view of a vascular access device according to a further embodiment after percutaneous insertion into the blood vessel;

FIG. 23b is a schematic side view of a variation of the extravascular part of the fixation mechanism of the vascular access device of FIG. 23a;

FIG. 23c is a schematic side view of another variation of the extravascular part of the fixation mechanism of the vascular access device of FIG. 23a;

FIG. 24a is a schematic side view of an extravascular part of the fixation mechanism of another embodiment of a vascular access device after percutaneous insertion into the blood vessel;

FIG. 24b is a schematic side view of the extravascular part of the fixation mechanism of the vascular access device of FIG. 24a;

FIG. 25a is a schematic side view of a vascular access device according to yet a further embodiment during insertion into the blood vessel;

FIG. 25b is a schematic side view of the vascular access device of FIG. 25a showing the intravascular part of the fixation mechanism during deployment;

FIG. 25c is a schematic side view of the vascular access device of FIG. 18a showing the intravascular part of the fixation mechanism after deployment;

FIG. 26 is a schematic side view of yet another embodiment of a vascular access device during insertion into the blood vessel;

FIG. 27a is a schematic side view of still a further embodiment of a vascular access device during insertion into the blood vessel;

FIG. 27b is a schematic side view of the vascular access device of FIG. 27a during deployment of the fixation mechanism;

FIG. 28a is a schematic side view of a vascular access system with a vascular access device in combination with a dilator during insertion into the blood vessel; and

FIG. 28b is a schematic side view of the vascular access system (device and dilator) of FIG. 28a during deployment of the fixation mechanism.

The accompanying drawings are included to provide a further understanding of the present invention and are incorporated in and constitute a part of this specification. The drawings illustrate particular embodiments and together with the description serve to explain the principles of the invention. Other embodiments and many of the attendant advantages will be readily appreciated as they become better understood with reference to the following detailed description.

It will be appreciated that common and/or well understood elements that may be useful or necessary in a commercially feasible embodiment are not necessarily depicted in order to facilitate a more abstracted view of the embodiments. The elements of the drawings are not necessarily illustrated to scale relative to each other. It will also be understood that certain actions and/or steps in an embodiment of a method may be described or depicted in a particular order of occurrences while those skilled in the art will understand that such specificity with respect to sequence is not actually required.

### DETAILED DESCRIPTION OF THE EMBODIMENTS

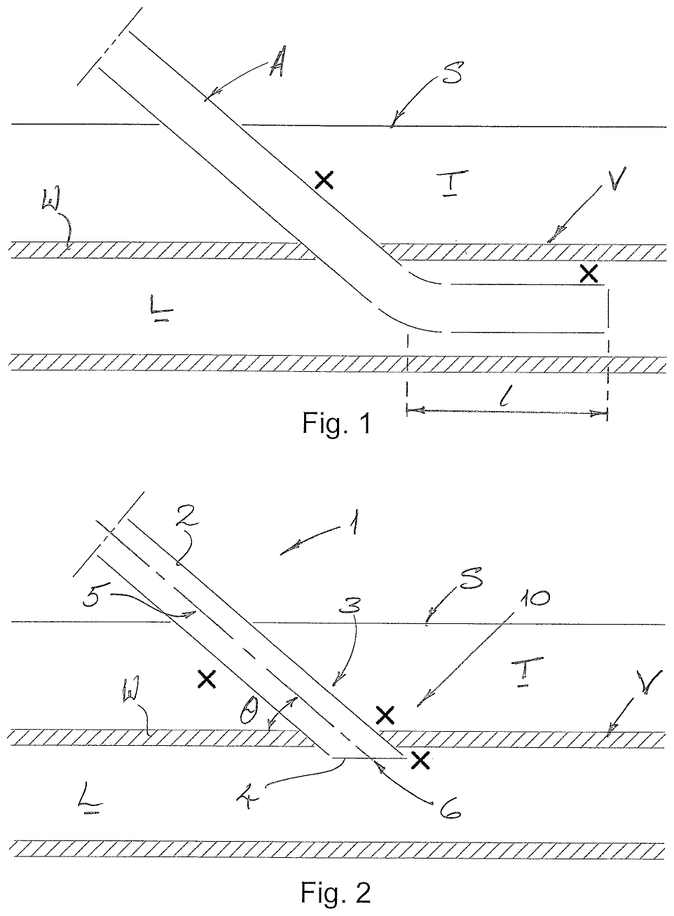

Referring firstly to FIG. 1 of the drawings, a cross-sectional view of a known vascular access tube A is shown in an inserted state extending within a blood vessel V. The vascular access tube A in this case was inserted percutaneously through the skin S and subcutaneous tissue T of the patient to penetrate the wall W of the blood vessel V such that an appreciable length 1 of the access tube A protrudes into and aligns with or extends along the lumen L of the blood vessel V. As noted above, however, this has the disadvantage that the length 1 of the access tube A residing in the blood vessel restricts blood flow through the blood vessel V and creates significant thrombotic/thrombo-embolic, ischaemic and intimal hyperplasia risks while the access tube A is in situ. The surfaces of the length 1 of access tube A also present a colonisation risk and possible infection resulting from such a colonisation.

With reference now to drawing FIG. 2, an example of a percutaneous vascular access device 1 illustrating the basic concept of the invention is shown. In this case, the vascular access device 1 comprises a vascular access tube 2, e.g. a cannula, having a distal end region 3 terminating in a tip 4 that is inserted into the blood vessel V of a patient and having at least one lumen 5 for infusing medicament and/or for introducing one or more catheters (not shown) there-through into the blood vessel V. In this regard, the access tube or cannula 2 has one or more opening 6 at its distal tip 4 to provide fluid communication between the at least one lumen 5 of the access tube 2 and the lumen L of the blood vessel V into which the tip 4 is inserted. The vascular access device 1 further includes a fixation mechanism 10 (identified only generically) that is operable to secure or fix the distal end region 3 of the vascular access tube 2 with respect to the patient. That is, the fixation mechanism 10 is configured to secure or fix the tip 4 (and thus the opening 6) of the vascular access tube 2 within the blood vessel V positioned at a location adjacent a wall W of the blood vessel at a location where the tip is inserted; i.e. with minimal intrusion into the vessel lumen L. By engaging with an inner surface of the blood vessel wall W, and/or an outer surface of the blood vessel wall W, and/or the subcutaneous tissue T at the positions marked “X”, the fixation mechanism 10 operates or acts to inhibit or prevent withdrawal of the tip 4 from the blood vessel V and/or to inhibit or prevent over-insertion of the tip 4 into the blood vessel V. Further, the fixation mechanism 10 secures or fixes the access tube 2 such that it extends longitudinally at a predetermined angle θ of about 45 degrees with respect to the blood vessel V at the point of insertion.

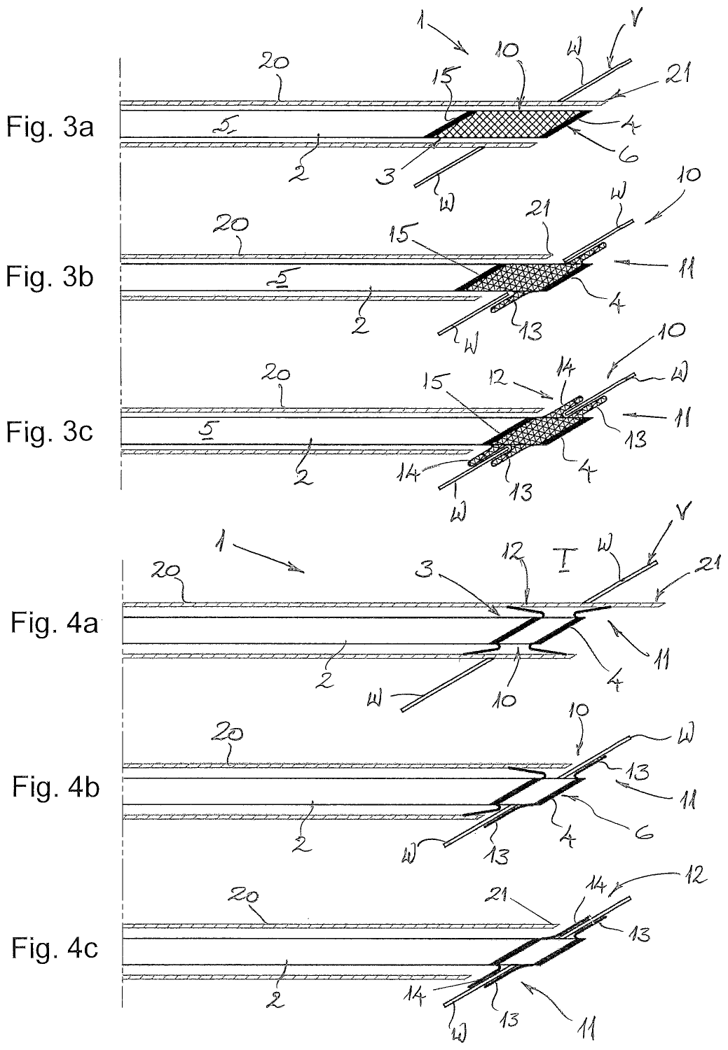

With reference then to FIGS. 3a to 3c of the drawings, a first embodiment of a percutaneous vascular access device 1 according to the invention is shown in various stages of insertion and deployment. In this embodiment, the vascular access device 1 is seen to include a cannula or vascular access tube 2, as noted above, but also includes a delivery sheath 20 for assisting percutaneous insertion or introduction and positioning of the vascular access tube 2 relative to the blood vessel V. The vascular access tube 2 is introduced via the delivery sheath 20, which has a distal tip 21 inserted through the skin S and subcutaneous tissue T of the patient to facilitate and guide the insertion of the tip 4 of the access tube 2 through the wall W of the blood vessel V, as shown in FIG. 3a. The distal end region 3 of the access tube or cannula 2 adjacent the tip 4 has a fixation mechanism 10, the operation of which becomes apparent from FIGS. 3b and 3c.

Referring to FIGS. 3b and 3c, the fixation mechanism 10 of this first embodiment includes an intravascular part 11 and an extravascular part 12. The intravascular part 11 comprises a member or element 13 in the form of a disc or flange comprising a covered mesh structure overlying the distal end region 3 adjacent the tip 4 of the cannula 2. The disc or flange member or element 13 is movable between a retracted or non-deployed position, as shown in FIG. 3a, where it is collapsed to lie flat against the transcutaneous and trans-vascular cannula 2 of the device 1 during introduction via the delivery sheath 20 for insertion of the tip 4 through the vascular wall W, and an extended or deployed position, as shown in FIG. 3b, for engaging an inner surface of the wall W of the blood vessel V to inhibit or prevent withdrawal of the tip 4. To this end, the disc or flange member 13 of the intravascular part 11 projects laterally or radially outward from the vascular access tube 2 in its extended or deployed position. Similarly, the extravascular part 12 comprises a member or element 14 formed as a disc or flange comprising a covered mesh structure which overlies the distal end region 3 adjacent the tip 4 of the cannula 2. The disc or flange member 14 is again movable between a retracted or non-deployed position, as shown in FIG. 3b, where it is collapsed to lie flat against the distal end region 3 of the transcutaneous and trans-vascular cannula 2 during introduction via the delivery sheath 20, and an extended or deployed position, as shown in FIG. 3c, for engaging an outer surface of the wall W of the blood vessel V to inhibit or prevent over-insertion of the tip 4. Thus, the extravascular part 12 is configured to cooperate with the intravascular part 11 of the fixation mechanism 10 to capture and clamp or sandwich the wall W of the blood vessel V there-between. In this way, the fixation mechanism 10 can be configured to secure or fix the access tube 2 so that it extends at a predetermined angle θ of about 40 degrees with respect to the blood vessel V at the point of insertion.

The disc or flange members or elements 13, 14 may be either (i) self-deploying, utilising a resilient bias or elastic recoil of the mesh structure to cause deployment into the disc or flange configuration as the sheath 20 is withdrawn; or (ii) manually deployed, utilising an axial force applied to the mesh structure to cause deployment into the disc configuration, with the axial force applied using a rigid (e.g. a tubular or partially tubular) activation member (not shown) and axially slidable collar 15 around the exterior surface of the access tube or cannula 2 of the device 1. Deployment is achieved in the first case by incrementally withdrawing the delivery sheath 20 used to introduce the device 1 until the intravascular disc member 13 of the fixation mechanism 10 deploys, then applying light traction to abut the disc member 13 against the inside of vascular wall W, and further incrementally withdrawing the sheath 20 while maintaining the traction until the extravascular disc member 14 deploys. In the second case, the deployment is achieved by carefully positioning the access device 1 so that the mesh structure of the fixation mechanism 10 spans the vascular wall W, then withdrawing the delivery sheath 20 to expose the fixation members 13, 14 (i.e. without displacing the device 1), and using the activation member (not shown) connected to the collar 15 surrounding an exterior surface of cannula 2 to apply an axial force to the fixation members 13, 14 to cause deployment. In both cases, the delivery sheath 20 is withdrawn entirely after the deployment of the fixation mechanism 10. A tearaway sheath 20 may be used to allow the sheath 20 to clear external elements of the device 1, depending on the geometry of those elements.

During percutaneous explantation, the mesh structure of the disc members 13, 14 of the fixation mechanism 10 can be re-collapsed to lie flat against the access tube or cannula 2 of the device 1. This may be achieved by using an axial force applied to the mesh structure (e.g. via the collar 15 and either a rigid activation member, such as the tubular member, or a flexible tension line, attached to the collar 15) to draw the fixation mechanism 10 (back) into a collapsed configuration. Alternatively, this may be achieved by passing a sheath 20 (e.g. a rigid sheath) over the external surface of the device 1 to pass over the disc members 13, 14 and thereby cause them to collapse down under the sheath. After collapse of the disc members 13, 14, the device 1 and surrounding sheath 20 can then be withdrawn, and a percutaneous arterial closure system is employed to seal the residual arterial puncture. It will be appreciated that the mesh-like disc or flange members 13, 14 of this embodiment may be used or employed individually in other embodiments—i.e. just an intravascular disc or flange members 13, optionally combined with some other extravascular part 12, or just an extravascular disc or flange members 14, optionally combined with some other intravascular part 11. The mesh-like disc or flange members 13, 14 may also include a flexible membrane or web covering.

With reference now to FIGS. 4a to 4c of the drawings, a second embodiment of a percutaneous vascular access device 1 according to the invention is shown in various stages of insertion and deployment. In this embodiment, the intravascular part 11 and the extravascular part 12 again comprise respective flange members 13, 14 collapsed or confined to lie flat against the access tube or cannula 2 of the device 1 in a retracted or non-deployed position within the delivery sheath 20, as shown in FIG. 4a. In this case, the flange members 13, 14 may again be biased or configured to move automatically to an extended or deployed position, as respectively seen in FIG. 4b or 4c. To this end, the flange members 13, 14 may be formed of Nitinol or some other super-elastic material. Deployment may thus be achieved by incrementally withdrawing the delivery sheath 20 used to introduce the device 1 through the skin S, the subcutaneous tissue T and into the blood vessel V until the intravascular flange member 13 of the fixation mechanism 10 deploys, then applying light traction to oppose that flange member 13 against the inside of the vascular wall W, and further incrementally withdrawing the sheath 20 while maintaining traction until the extravascular flange member 14 deploys.

If the flange members 13, 14 were not configured to deploy automatically upon release from the confinement of the sheath 20, the deployment could alternatively be achieved in a second case by incrementally withdrawing the delivery sheath 20 used to introduce the access tube or cannula 2 until the collapsed intravascular flange 13 of the fixation mechanism 10 is exposed within the vessel V. A balloon catheter (not shown) would then be positioned adjacent to the flange member 13 and inflated to deform the collapsed flange member 13 into its deployed configuration, as shown in FIG. 4b. A light traction is applied to oppose the flange member 13 against the vascular wall W, and the delivery sheath 20 is further incrementally withdrawn while maintaining traction until the extravascular flange member 14 is released. If the flange member 14 were also not self-deploying, it may likewise be deformed to the position shown in FIG. 4c (e.g. by a collar, tubular, or split tubular member slidable or movable on an outer periphery of the access tube or cannula 2) to engage an outer surface of the vessel wall W. In both cases, the sheath 20 is withdrawn completely after deployment of the flange members 13, 14. As before, during percutaneous explantation, the flange members 13, 14 of the fixation mechanism 10 are moved back to a retracted or collapsed position against the cannula 2 of the device 1. After collapse of the flange members 13, 14, the access device 1 and the confining sheath 20 are withdrawn, and a percutaneous arterial closure system may be employed to seal the residual vascular puncture.

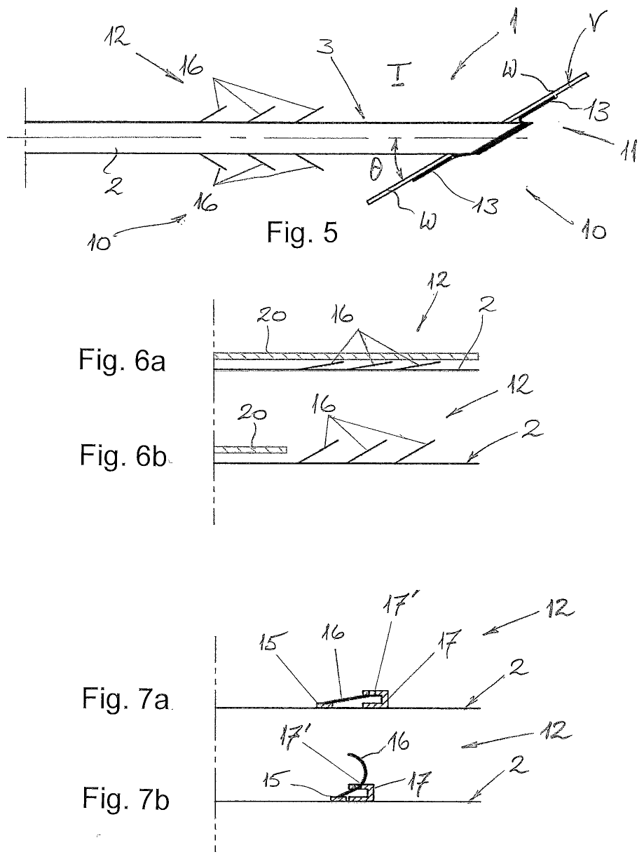

With reference now to drawing FIGS. 5, 6a, 6b, 7a and 7b, other embodiments of a percutaneous vascular access device 1 are shown in different stages of insertion and deployment. That is, FIG. 5 shows a schematic side view of a vascular access device 1 according to a third embodiment with the tip 4 of the access tube or cannula 2 inserted and secured in a blood vessel V with fixation mechanism 10 activated. The intravascular part 11 again comprises a disc or flange member 13 like the embodiments described above. In this case, however, the extravascular part 12 does not have a corresponding disc or flange member 14. Rather, the extravascular part 12 comprises a set of prongs or elongate barbs 16 that are outwardly biased to project into and engage subcutaneous tissue T of the patient in their deployed position, as shown. In this way, these angled prongs or barbs 16 act to resist or inhibit further movement of the access tube 2 (and, thus, the tip 4) towards or into the blood vessel V. FIG. 6a shows detail of the prongs or elongate barbs 16 confined or held in their retracted or non-deployed position within the delivery sheath 20, and FIG. 6b shows the prongs or barbs 16 released as the sheath 20 is withdrawn. In this embodiment, the prongs or barbs 16 are biased outwardly from the sides of the access tube or cannula 2 in a resilient or elastic manner so that they deploy automatically upon withdrawal of the delivery sheath 20.

Drawing FIGS. 7a and 7b show a variation (fourth embodiment), however, where the movement of the prongs or barbs 16 are controlled by activation members 15, 17 of the fixation mechanism 10. In particular, a proximal end of each of the barbs 16 is attached to a slidable collar 15 on an outer periphery of the vascular access tube or cannula 2, as seen in FIG. 7a. An opposite end of each barb 16 is guided through a channel 17′ formed in a guide ring 17 fixed on an outer periphery of the vascular access tube or cannula 2. In this way, when the collar 15 is slid towards the guide ring 17, as seen in FIG. 7b, each prong or barb 16 is driven through the guide channel 17′, which directs the prong or barb 16 radially outwards into the subcutaneous tissue T into the deployed position. Again, therefore, these outwardly directed prongs or barbs 16 act to resist or inhibit further movement of the access tube 2 towards or into the blood vessel V, thereby inhibiting or preventing over-insertion of the tip 4. During the percutaneous removal or explantation of this vascular access device 1, the prongs or barbs 16 of the fixation mechanism 10 can be retracted or collapsed to lie flat against the access tube 2. That is, the actuated barbs 16 described above can be retracted or collapsed by sliding the collar 15 away from the guide ring 17, back to the position shown in FIG. 7a. In the case of FIG. 5, the retraction may be achieved by passing one or more sheaths 20 back over the exterior of the access tube 2 to cause the barbs 16 to collapse down within the sheath 20, i.e. back to the position shown in FIG. 6a. Although the collar 15 is described as being axially slidable relative to the fixed guide ring 17 in FIGS. 7a and 7b, it will be appreciated that the guide ring 17 may alternatively be axially slidable relative to the collar 15 to achieve the same result. It will also be appreciated that the prongs or barbs 16 may be configured or arranged in a variety of ways to be extended from a non-deployed position in the access tube 2 to project radially outwardly into subcutaneous tissue T in a deployed position. For example, the elongate prongs or barbs 16 in FIG. 5 could be mounted on the ends of pivot arms, and/or could be configured or arranged to be driven outwards upon the insertion (e.g. axially) or operation of an activation member within the access tube 2. In this regard, the activation member could be in the form of an internal sleeve or tube (not shown) that is axially slidable within the lumen 5, e.g. like a liner of the access tube or cannula 2. It could also be in the form of an axially slidable rod or element housed within channels in a wall of the access tube or cannula 2, seen in later embodiments. The elongate prongs or barbs 16 could thus be driven out laterally (e.g. radially) via apertures in the side walls of the tube or cannula 2 when the activation member(s) is/are advanced distally along the axial channel(s). The activation member could also be in the form of an inflatable balloon member (not shown). The elongate prongs or barbs 16 could be arranged at an inclined angle to the access tube 2, i.e. as shown in FIG. 5, or they could project radially outwards at 90 degrees to the axis of the access tube 2. Importantly, the direction of the elongate prongs or barbs 16 and their paths of deployment through the subcutaneous tissue T can be modified by adjusting their geometry and the deployment mechanisms (e.g. by altering the shape and direction of apertures in cannula walls through which the elements deploy, or the shape of cantilever arms on which they are mounted etc.).

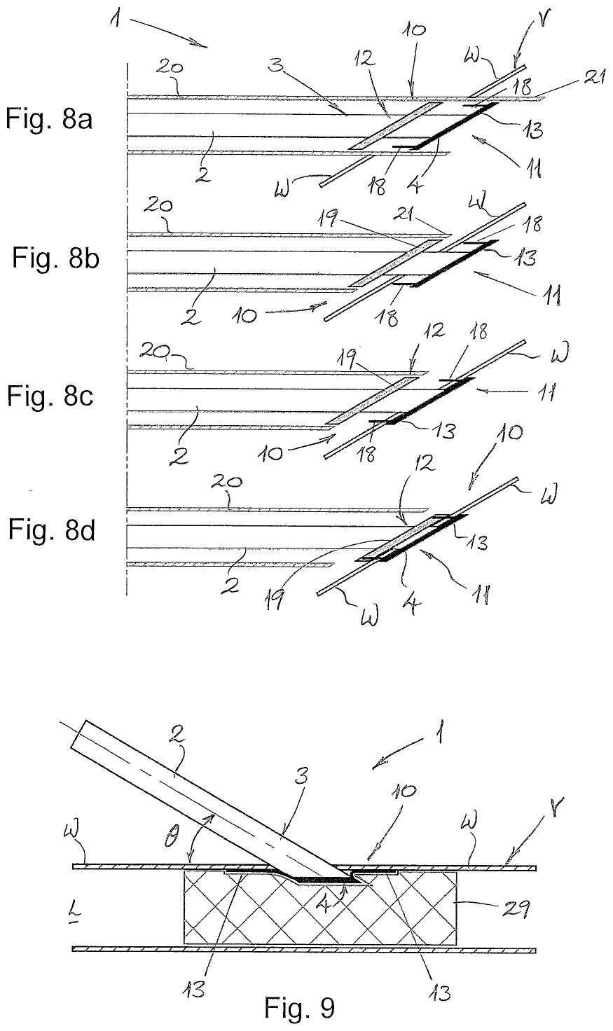

Referring to FIGS. 8a to 8d of the drawings, a fifth embodiment of a vascular access device 1 is shown in various stages of insertion and deployment. It will be noted that the vascular access device 1 in this case has an intravascular part 11 comprising a non-collapsible, substantially rigid flange member 13. For this reason, an inner diameter of the delivery sheath 20 is necessarily somewhat larger to accommodate the flange member 13. Also, it will be noted that the flange member 13 includes upstanding prongs or barbs 18. The extravascular part 12 of this embodiment is in the form of an axially slidable collar 19 having approximately the same diameter as the flange member 13. As in the previously described embodiments, the vascular access device 1 of FIGS. 8a to 8d is percutaneously introduced or implanted in a patient via a delivery sheath 20. In this case, because the delivery sheath 20 is of substantially larger diameter than the access tube 2, the breach or puncture through the wall W of the blood vessel V is significantly dilated by the sheath 20. As the sheath 20 is withdrawn from the position in FIG. 8a out of the vessel wall W to the position in FIG. 8b during deployment of the device 1, the breach or puncture through the wall W contracts around the access tube 2 such that it can be engaged by the intravascular flange member 13. Light traction is applied to pull the rigid flange 13 against the vascular wall W and cause the axially directed barbs 18 to penetrate through the vascular wall W, as shown in FIG. 8c. Using a tubular or partly tubular member (not shown) around the exterior of the transcutaneous access tube or cannula 2, axial force is applied to displace the slidable collar 19 of the extravascular part 12 towards the vessel wall W such that the barbs or prongs 18 protruding from the wall W penetrate and are retained by the collar 19. In this way, the wall W of the blood vessel V is effectively clamped and captured or sandwiched between the intravascular and extra-vascular parts 11, 12 of the fixation mechanism 10, as shown in FIG. 8d.

It will be appreciated that the intravascular and extravascular parts 11, 12 of the fixation mechanism 10 of this embodiment could be reversed without any substantial change of operation. That is, the slidable collar 19 could include axial prongs or barbs and the flange 13 could be able to be penetrated or pierced by those prongs or barbs. During percutaneous explantation of the vascular access device 1 of FIGS. 8a to 8d, the slidable collar 19 is retracted using a tubular or split tubular member (not shown) on the outside of the access tube 2 that is able to interlock with the collar 19 to apply tension, or by using a tether or tension line attached to the collar 19 itself. After withdrawing the collar 19, one or more sheaths 20 (in incrementally increasing diameters) are passed over an outer surface of the access tube 2 to provide a sheath 20 encompassing the intravascular flange member 13. The device 1 and sheath 20 can then be withdrawn from the blood vessel V, and a percutaneous arterial closure system can be employed to seal the residual arterial puncture.

With reference now to drawing FIG. 9, a sixth embodiment of a percutaneous vascular access device 1 is shown in a deployed state. In this embodiment, in addition to a flange member 13, the intra-vascular part 11 of fixation mechanism 10 comprises a stent member 29. During deployment, the stent 29 is collapsed and is introduced into the blood vessel V using a catheter via a separate access site, or through the lumen 5 of the access tube or cannula 2 of the device 1 itself. The intravascular stent 29 of this fixation mechanism 10 may be either: (i) self-deploying, where the stent 29 is collapsed inside a confining catheter, with the stent deploying via elastic recoil of its structure after removal of said confining catheter; or (ii) balloon deployed, where the stent member 29 is mounted onto a balloon catheter (not shown) and where pressurisation of the balloon catheter is used to apply load to plastically deform the stent 29 into its deployed position and configuration, as is understood in the art. In either case, during deployment of the stent 29: traction is applied and maintained to pull the flange member 13 of the fixation mechanism 10 against the vascular wall W. The intravascular stent 29 is introduced via a separate vascular access, or via the lumen 5 of the cannula 2 of the device 1, and positioned in the part of the blood vessel V where the intravascular flange member 13 engages against the vascular wall W. The intravascular stent 29, when deployed as described, presses the intravascular flange member 13 against the vascular wall W, after which traction on the device 1 can be released. This embodiment of the vascular access device 1 differs from the other embodiments described above in that members 13,29 of the intravascular part 11 operate to inhibit or prevent both an inadvertent withdrawal of the tip 4 of the access tube 2 from the blood vessel V as well as an over-insertion of the tip 4 into the blood vessel V.

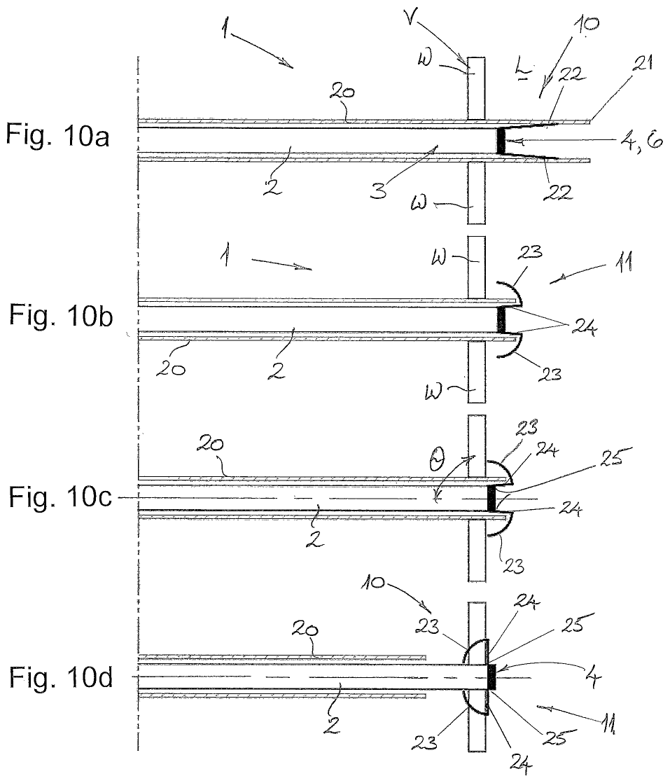

With reference now to FIGS. 10a to 10d of the drawings, a seventh embodiment of a percutaneous vascular access device 1 is shown in various stages of insertion and deployment. In this embodiment, the intravascular part 11 of the fixation mechanism 10 comprises a set (two or more) of self-deploying and bidirectionally fixating members 22 attached at the tip 4 of the access tube or cannula 2. Each of these fixating members 22 comprises a prong or barb 23 formed as a flexible, deformable circular or near-circular arc segment that extends substantially perpendicularly from an end of a straight arm segment 24, which in turn is connected to the tip 4 of the access tube 2 via an elastic hinge point 25 around which a circular or near-circular motion of the prong or barb 23 is produced. Each of the prongs or barbs 23 has a sharp point able to penetrate through the wall W of the blood vessel V and a radius equal or near-equal to the length of the straight arm segment 24. These fixating members 22 are typically evenly spaced apart around the periphery of the tip 4.

During percutaneous introduction of the vascular access device 1 through the skin S, subcutaneous tissue T, and vascular wall W via a delivery sheath 20, as shown in FIG. 10a, the fixating members 22 are in a collapsed or straightened, non-deployed position lying flat against the delivery sheath 20. Deployment of the fixating members 22 is achieved by incrementally withdrawing the delivery sheath 20 until the circular or near-circular arc segments of the prongs or barbs 23 are released and recover to their intended geometry by elastic recoil, as shown in FIG. 10b. Traction is then applied to the access tube 2 to cause the sharp points of the prongs or barbs 23 to contact and just shallowly pierce the vascular wall W, as shown in FIG. 10c. Maintaining traction, the delivery sheath 20 is fully withdrawn from the blood vessel V and, as the sheath 20 clears the elastic hinge points 25 of the fixating members 22, the straight arms 24 rotate to their intended positions by via the elastic hinge points, thereby causing the circular or near-circular arc segments of the prongs or barbs 23 to rotate around this hinge point and to pierce smoothly through the tissue of the vascular wall W, such that the fixating members 22 deploy fully with the straight arm segments 24 engaging the wall W, as shown in FIG. 10d. In this embodiment, the access tube or cannula 2 is fixed or secured by the fixation mechanism 10 such that it extends at a predetermined angle θ of about 90 degrees with respect to the blood vessel V at the point of insertion of the tip 4.

In the deployed position, the fixating members 22 capture or grasp a volume of tissue between the circular or near-circular arc segments of the prongs or barbs 23 and the straight arm segments 24. Furthermore, the motion of the fixating members 22 during deployment occurs independently of any axial motion of the access tube or cannula 2, resulting in no ‘natural’ exit path for release of the fixating members 22 in the scenario where an axial force is applied to the device 1 (i.e. either for withdrawal or for over-insertion of the tip of the access tube 2). As a result, the fixating members 22 are able to produce a bidirectional fixation of the transcutaneous access tube or cannula 2 to the wall W of the blood vessel V. After deployment of the fixation mechanism 10, the delivery sheath 20 is typically withdrawn completely. During percutaneous explantation of the vascular access device 1, the fixating members 22 of the fixation mechanism 10 are re-collapsed to lie flat in the non-deployed position by passing a sheath 20 over the outer surface of the access tube 2 to cause them to collapse down again within the sheath 20, as in FIG. 10a. After collapsing the fixating members 22, the device 1 and the sheath 20 may be withdrawn, and a percutaneous arterial closure system may be employed to seal the residual arterial puncture.

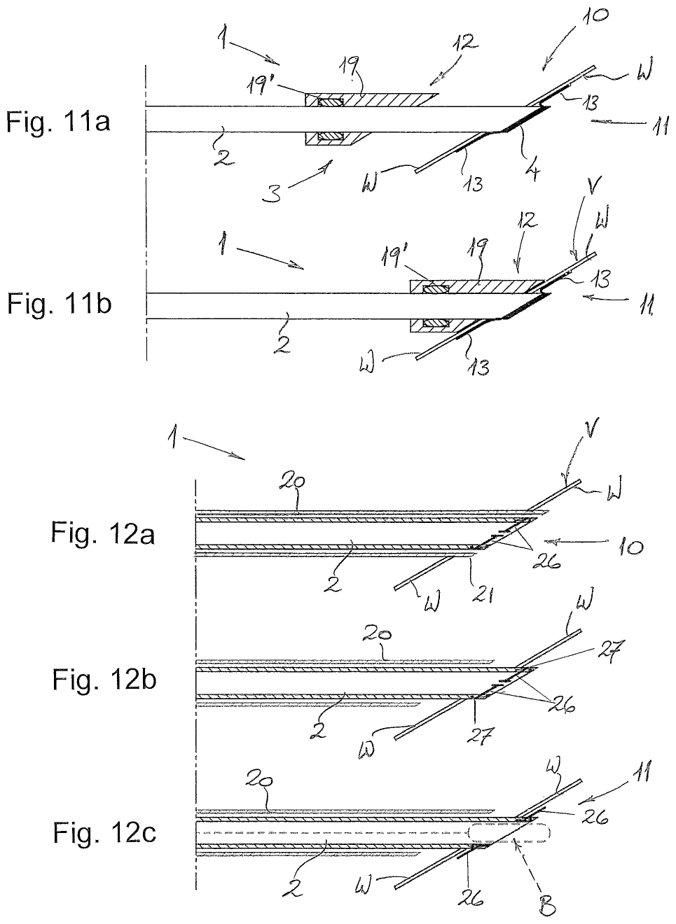

Referring to drawing FIGS. 11a and 11b, an eighth embodiment of a vascular access device 1 is shown in two stages of deployment. This embodiment is basically a modification of the second embodiment of FIGS. 4a to 4c, in which the flange member 14 of the extravascular part 12 is replaced with an axially slidable collar member 19 (i e similar to the embodiment of FIGS. 8a to 8d) encircling an outer surface of the access tube 2 of the device 1, and interfacing with that outer surface via a bearing or seal (e.g. an O-ring) 19′ having a pre-calibrated magnitude of dynamic and static friction. During introduction of the vascular access device 1 via a delivery sheath 20 (i.e. as described for the second embodiment), the slidable collar member 19 is located at the proximal (extracorporeal) end region of the access tube or cannula 2. Then, following deployment of the intravascular flange member 13 and after removal of the delivery sheath 20, traction is applied to pull the intravascular flange member 13 of the fixation mechanism 10 against the inner side of vascular wall W and the extravascular collar member 19 is pushed distally to a point of entry of the device 1 through the skin. Thereafter, a tubular, partial tubular or other shaped pusher member (not shown) that fits around or adjacent to the access tube or cannula 2 is used to push the collar member 19 further distally along the length of the access tube 2 while maintaining traction until the collar member 19 meets the vascular wall W and clamps or sandwiches it against the intravascular flange member 13. During percutaneous explantation, the slidable collar member 19 is retracted along the outer surface of the access tube 2 using a retractor member (not shown) that can interlock with the collar 19 and apply tension, or by using a tether/tension line attached to the collar 19.

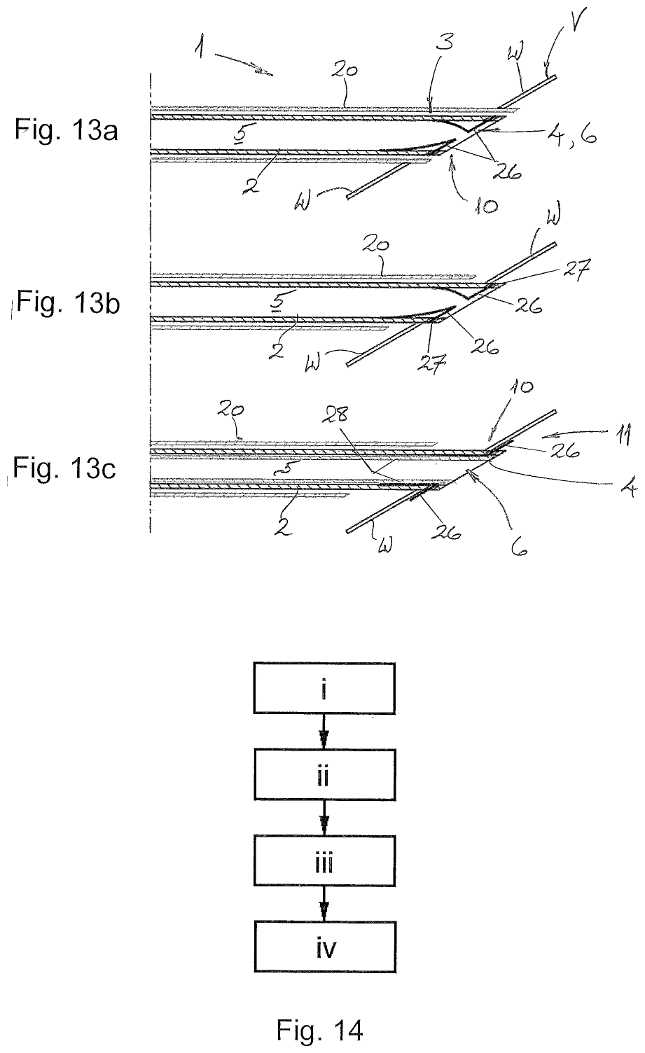

With reference now to FIGS. 12a to 12c and FIGS. 13a to 13c of the drawings, a ninth and a tenth embodiment of a percutaneous vascular access device 1 is shown in various stages of insertion and deployment. These two embodiments are variations of earlier embodiments, where the flange member 13 of the fixation mechanism 10 is now replaced by a set of (e.g. two, three, four, or more) arm members 26 that are retractable within the lumen 5 of the transcutaneous access tube or cannula 2 of the device 1. That is, rather than being collapsible against an outer surface of access tube or cannula 2, the retractable operation is accomplished by either: (i) mounting the arm members 26 for deployment (e.g. via sliding movement) through respective apertures, e.g. slots, 27 in the sides of the access tube 2 in the embodiment of FIGS. 12a to 12c; or (ii) mounting arm members 26 for deployment (e.g. via pivoting movement) in an internal cantilevered configuration, with ends of the cantilevered arms either protruding beyond the tip 4 of access tube 2, or passing through corresponding apertures, e.g. slots, 27 in the sides of the access tube 2, as per the embodiment of FIGS. 13a to 13c. In both cases, during the introduction of the vascular access device 1 into the patient, the arm members 26 are in a retracted or non-deployed position (as shown in FIG. 12a and FIG. 13a) to facilitate percutaneous insertion. Depending on the degree of retraction of the arm members 26, the device 1 may be introduced into the blood vessel V via a delivery sheath 20, or may be advanced directly over a guidewire (e.g. with the aid of a dilator) without an external sheath 20 in a variation of the Seldinger technique.

It will be appreciated that the “arm members” 26 in the above embodiments may be configured as relatively narrow rod-like or strip-like elements. Alternatively, however, they may also be formed as somewhat broader panel members or flap members. The arm members 26 may optionally also be interconnected by a flexible membrane or web (not shown) such that they together effectively define a continuous flange for abutting or engaging the wall of the blood vessel. This range of different possible implementations will be understood by skilled persons to apply for the “arm members” 26 described in any of the embodiments herein, unless the context indicates to the contrary.

In the event that a delivery sheath 20 is used, after the tip 4 of the access tube 2 is inserted through the wall W of the blood vessel V, the sheath 20 may be withdrawn from the blood vessel thereby causing the wall W to contract onto an outer surface of the access tube 2 and also opening the slots 27 for passage of the arm members 26 there-through, as seen in FIG. 12b and FIG. 13b. Deployment of the arm members 26 in the case “(i)” may be achieved by inflating a vascular balloon B inside the lumen 5 of the access tube 2, as shown in FIG. 12c. The force applied by the balloon causes the arm members 26 to be actuated outwards through their corresponding slots 27 in the access tube or cannula 2 of the device 1 to form externally protruding structure. In this regard, the arm members 26 may be held by friction or by a detent in the slots 27 to retain this externally protruding position, until they are retracted. Deployment of the arm members 26 in the case “(ii)” can be achieved by advancing an internal cannula element 28, e.g. as a sleeve or liner, through the lumen 5 of the access tube or cannula 2, with the force applied by advancement of the internal cannula element 28 actuating the cantilevered arm members 26 outward to form an externally protruding structure. As an alternative, a vascular balloon B could again be used, if the cantilevered arm members 26 deformed plastically or latched to maintain the externally protruding position. During percutaneous explantation of the access device 1, the arm members 26 of the fixation mechanism 10 are retracted back into the lumen 5 of the access tube 2. This could, for example, be achieved for the arm members 26 in case “(ii)”—i.e. FIGS. 13a to 13c—by retracting the internal cannula element or sleeve 28, causing the cantilevered arm members 26 to return to the retracted positions under their own resilient bias or elastic recoil. If the arm members 26 had been plastically deformed or latched into their deployed position, they would need to be actively retracted back into the cannula 2 prior to explantation of the device 1, for example via a flexible activation member (e.g. a cord or line) to be drawn or pulled under tension by an operator at a proximal end region of the vascular access device 1.

Further, referring to FIG. 14 of the drawings, a flow diagram is shown to illustrate schematically the steps in a method of using a percutaneous vascular access device 1 according to the various embodiments of the invention described above with respect to FIGS. 3a to 13c. In this regard, the first box i of FIG. 14 represents the step of inserting a vascular access tube 2 percutaneously into a patient, the access tube 2 having a tip 4 for insertion through a wall W of a blood vessel V of the patient and at least one lumen 5 for introducing one or more catheters there-through into the blood vessel V. The second box ii then represents the step of activating a fixation mechanism 10 provided at a distal end region 3 of the vascular access tube 2 adjacent to the tip 4 to secure or fix the distal end region of the access tube 2 with respect to the patient, according to any one of the embodiments of the invention described above. In this way, the fixation mechanism 10 secures or fixes the tip 4 of the access tube 2 at or adjacent the wall W of the blood vessel V at a point of entry or insertion through the wall W such that an opening 6 in the tip 4 for providing fluid communication between the lumen 5 of the access tube 2 and the blood vessel V is positioned and secured or fixed at or adjacent the vessel wall W at the point of insertion of the tip 4. The third box iii represents the step of generalised in-situ use of the access tube 2, during which multiple procedures involving infusion and/or aspiration and/or introduction of catheters can be performed through the lumen 5 of the access tube 2 into the blood vessel V during treatment of the patient. The lumen 5 of the access tube 2 can be closed between procedures by an occluder member or by fluid locking during the in-situ use of the device 1. The final box iv in FIG. 14 of the drawings represents the step of subsequently deactivating the fixation mechanism 10 at the distal end region 3 of the vascular access tube 2 adjacent the tip 4 to release the distal end region 3 of the access tube 2 with respect to the patient for percutaneous removal or explantation of the device 1.

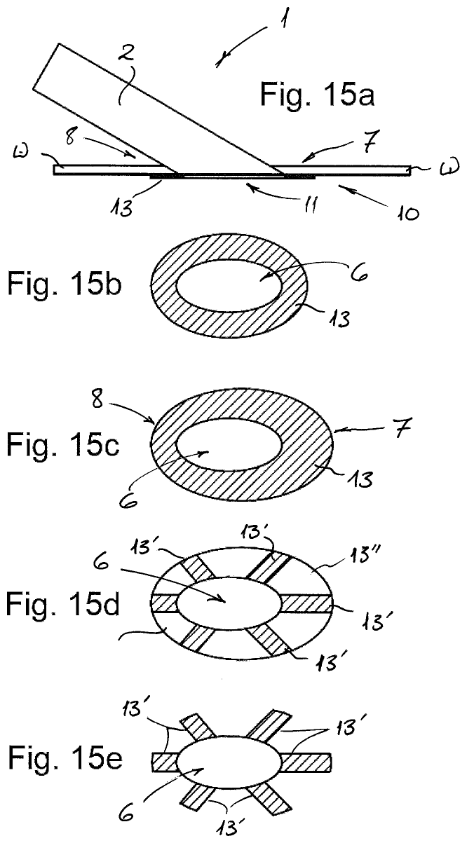

With reference now to FIGS. 15a to 15e of the drawings, examples of possible flange configurations are shown for embodiments in which the intravascular part 11 of the fixation mechanism 10 comprises a flange member 13. In this regard, FIG. 15a shows an arrangement of the vascular access device 1 with the cannula 2 and fixation mechanism 10 positioned relative to the vessel wall W for reference purposes. Drawing FIGS. 15b-15e show projected views of the flange member 13 of the intravascular part in various possible forms. It will be noted that the cannula 2 appears to have an elliptical cross-section with the major axis of the ellipse configured to be oriented longitudinally of the blood vessel V. In fact, the cannula 2 may have a basic circular cross-section but the angled orientation of the opening 6 creates this elliptical cross-section in projection. It is conceivable, however, that the cannula 2 could have an elliptical cross section in this manner to provide an enlarged space for accommodating catheters without unduly stressing the vessel wall W. FIG. 15b of the drawings shows a continuous flange member 13 that extends uniformly around a periphery of the tip 4 of the cannula 2. Drawing FIG. 15c, by contrast, shows a continuous flange member 13 that extends with non-uniform geometry around a periphery of the tip 4 of the cannula 2 with greater radial extent at a “toe” region 7 of the cannula 2 where a higher propensity for the cannula 2 to disengage from the vessel wall W may exist under application of a pulling force (e.g. due to normal loads or an inadvertent knock) compared to an opposite “heel” region 8 of the cannula 2. Drawing FIG. 15d illustrates a segmented flange member 13 having a plurality of individual flange elements 13′, formed as panels or flaps, which are inter-connected by a webbing or membrane structure 13″. FIG. 15e of the drawings, on the other hand, shows a segmented flange member 13 comprising a plurality of the flange elements 13′ in the form of separate panels or flaps that are not interconnected.

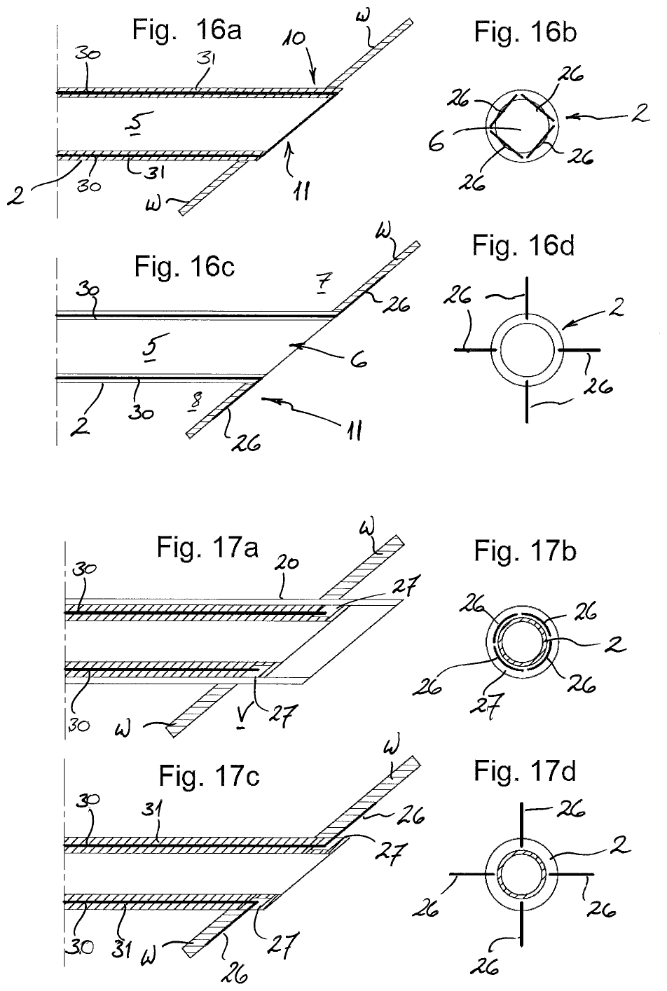

Referring to FIGS. 16a-16d of the drawings, another embodiment of a fixation mechanism 10 is illustrated in which the intravascular part 11 comprises a plurality of elongate members, such as arm members, 26 for engaging and bearing against an inner side of the vascular wall W. FIGS. 16a and 16b illustrate the fixation mechanism 10 in a non-deployed configuration, with each of the arm members 26 held in a retracted or non-deployed position against the distal tip 4 of the cannula 2. Each arm member 26 is mounted on an elongate rotatable actuating member 30, such as a torsion rod, that extends longitudinally within a channel 31 in the cannula wall. Each torsion rod 30 is, in turn, connected with a proximal handle or actuator (not shown) for operation by a medical practitioner performing the vascular access. Rotation of each torsion rod 30 actuates the respective arm member 26 for deployment. The drawing FIGS. 16c and 16d show the fixation mechanism 10 in a deployed configuration. In particular, the torsion rods 30 have been rotated, causing each arm member 26 of the intravascular part 11 to rotate into its extended or deployed position, with arm members 26 extending parallel to the flat chamfered tip 4 of the cannula 2 and lying opposed to the vessel wall W. The fixation mechanism 10 in this case does not rely on deformation. The arm members 26 may be retracted by rotating the torsion rods 30 in the reverse direction to return the arms 26 to their non-deployed positions. Alternatively, the arm members 26 could also be collapsed by advancing a sheath 20 around the outer surface of the cannula 2 to deform and collapse the arm members 26.

With reference to FIGS. 17a-17d of the drawings, another embodiment similar to the embodiment of FIGS. 16a-16d is shown with a fixation mechanism 10 in which the intravascular part 11 comprises a plurality of elongate arm members 26 for engaging and bearing against an inner side of the vascular wall W. In this embodiment, the arm members 26 are outwardly biased via the torsion rods 30 for deployment and are retained in the retracted position by a confining outer sheath 20, as shown in FIGS. 17a and 17b. In the retracted or non-deployed position, the arm members 26 are held within a recess or slot 27 extending around the circumference of the distal tip of the cannula, with each arm member 26 confined within the recess 27 of the cannula 2. The arm members 26 are resiliently biased via the respective torsion rods 30 to move towards the extended or deployed position when held in the retracted position. When the sheath 20 is withdrawn, thereby releasing the arm members 26 from their recesses 27 and allowing the torsion rods 30 to recover elastically, each of the arm members 26 is free to rotate into its extended or deployed position extending from the chamfered tip 4 of the cannula 2 and lying opposite the inner surface of the vessel wall W, as shown in FIGS. 17c and 17d. The arm members 26 may be retracted by rotating the torsion rods 30 in the reverse direction (i.e. against their resilient bias) to return the arms 26 to their non-deployed positions in the circumferential recess or slot 27. Alternatively, the arm members 26 could also be collapsed by advancing a sheath around the outer surface of the cannula 2 to deform and collapse the arm members.

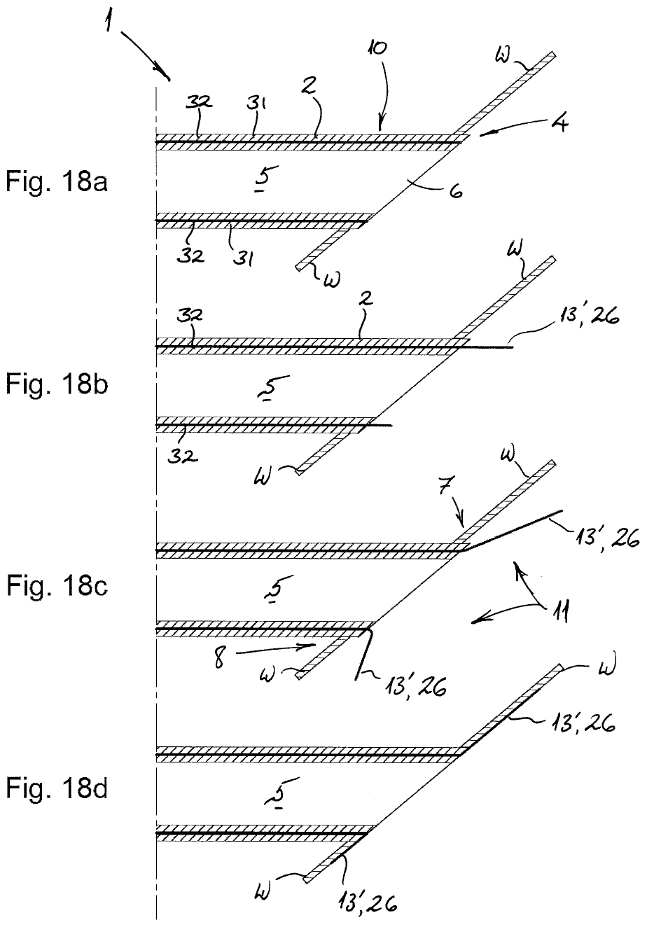

Referring to FIGS. 18a to 18d of the drawings, yet another embodiment of the fixation mechanism 10 is illustrated. In this example, the fixation mechanism 10 has an intravascular part 11 with one or more slidable actuating member(s) 32 for movement of flange elements 13′ or arm members 26 between a retracted, non-deployed position shown in FIG. 18a and an extended or deployed position shown in FIG. 18d. The flange elements 13′ or arm members 26 are housed within a channel 31 formed in the cannula wall and are continuous with or connected with the slidable actuating member(s) 32 also retained within that channel 31. FIGS. 18b and 18c of the drawings show the fixation mechanism 10 in intermediate states in the course of deployment as the axially slidable actuating member(s) 32 are advanced distally through the channel 31 in the wall of the cannula 2, causing the flange elements 13′ or arm members 26 to exit the channel 31 at the distal tip 4 of the cannula 2. As the flange elements 13′ or arm members 26 emerge from the channel 31 at the tip 4 of the cannula 2, they elastically recover to a final or deployed configuration, whereupon they bear against an inside of the vessel wall W, as shown in FIG. 18d, after the actuating member 32 has been fully advanced through the channel 31 in the cannula wall. Deformation of each flange element 13′ or arm member 26 is elastic such that the deployment is achieved by elastic recovery of the respective flange element 13′ or arm member 26 as it is advanced from the tip 4. Each flange element 13′ or arm member 26 may be retracted by pulling the axially slidable actuating member(s) 32 back in the proximal direction, thereby drawing the flange element 13′ or arm member 26 back into the channel 31 in the cannula wall. Alternatively, the flange elements 13′ or arm members 26 may be retracted by advancing a sheath (or series of sheaths) around the outer surface of the cannula 2 (and into the vessel V) to deform and collapse the flange elements 13′ or arm members 26 (not shown).

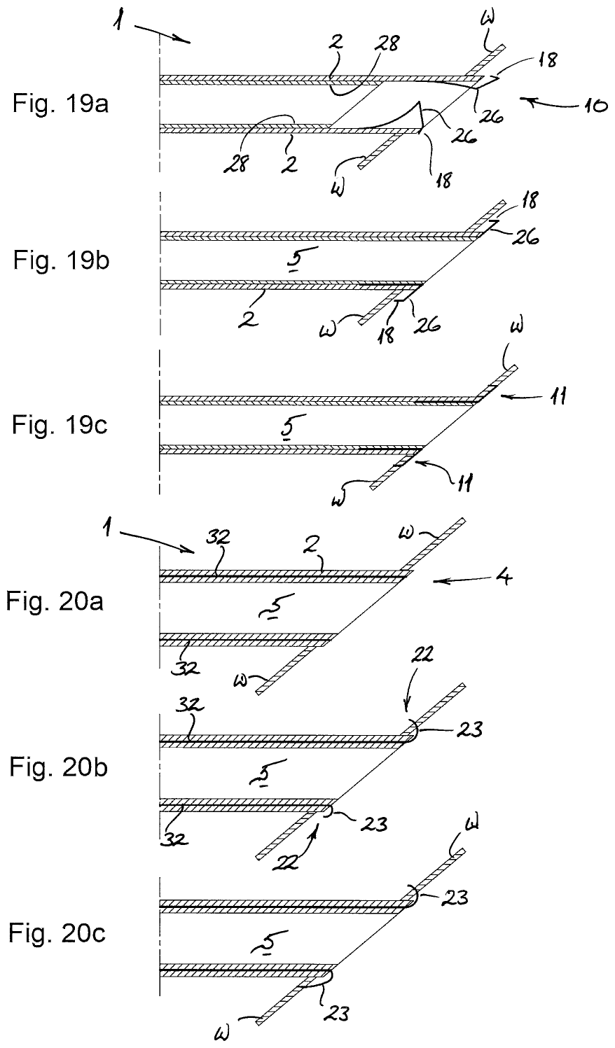

Referring to drawing FIGS. 19a to 19c, an embodiment of the vascular access device 1 analogous to that of FIGS. 13a to 13c is shown. In this case, the intravascular part 11 again comprises a plurality of cantilevered arm members 26, free ends of which include barbs 18. During introduction of the vascular access device 1 into the patient, the arm members 26 are in a retracted or non-deployed position within the lumen 5 of the cannula 2, as shown in FIG. 19a, to facilitate percutaneous insertion. As before, the device 1 may be introduced into the blood vessel V via a delivery sheath 20 or may be advanced directly over a guidewire (e.g. with the aid of a dilator) without an external sheath 20 in a variation of the Seldinger technique. Deployment of the arm members 26 is achieved by advancing an internal cannula element or sleeve 28 through the lumen 5 of the access tube or cannula 2. The force applied by the advancement of the internal cannula element 28 drives the cantilevered arm members 26 radially outwards from the opening 6 at the tip 4 of the cannula 2 to form an externally protruding structure as seen in FIG. 19b, with the barbs 18 projecting back towards the inside of the vascular wall W. By the operator then applying a light tension or pulling force to the access tube 2, the deployed arm members are drawn against wall W of the vessel, which the barbs 18 then pierce to fix or secure the vascular access device 1 with respect to the vessel V. From the example in FIGS. 19a to 19c, it will be understood by skilled persons that many embodiments of the intravascular part 11 of the fixation mechanism 10 may be modified or augmented by the inclusion of an active fixation element like barb 18. Depending on the specific implementation, the role of such barbs 18 may range from just augmenting engagement of the intravascular part 11 with the vessel wall W to providing a primary means of fixation.

With reference to drawing FIGS. 20a to 20c, another embodiment of a vascular access device 1 with a fixation mechanism 10 having some similarity to the embodiment of FIGS. 18a to 18c is shown. In this embodiment, the fixation mechanism 10 again has one or more slidable actuating member(s) 32 provided in channels 31 formed in a wall of the access tube/cannula 2 for moving the intravascular part 11 between a retracted, non-deployed position shown in FIG. 20a and an extended or deployed position shown in FIG. 20c. In this case, however, the intravascular part 11 of the fixation mechanism 10 comprises a set (two or more) of self-deploying fixating members 22 at an end of the actuating member(s) 32 similar to the embodiment of FIGS. 10a to 10c. Each fixating member 22 comprises a prong or barb 23 formed as a flexible arc segment that, during advancement of the axially slidable actuating member(s) 32, extends outwards from the tip 4 of the cannula 3 as shown in FIGS. 20b and 20c, and curves back towards the wall W of the blood vessel V with a sharp point able to penetrate through the wall W. These fixating members 22 are typically evenly spaced apart around the periphery of the tip 4. Thus, it will be appreciated that the present disclosure contemplates two different types of prong or barb elements 18, 23; namely: simple retrograde barbs, and bi-directionally fixating barbs having an arc or bent shape. In the case of bi-directionally fixating prongs or barbs: deliberate shaping of the prongs or barbs 18, 23 and deliberate design of the mechanisms involved in their deployment (such as the guide ring 17 and channel 17′ in FIGS. 7a and 7b, or the pivoting arms 24 in FIGS. 10a to 10d, or the axially slidable actuating member(s) 32 in FIGS. 20a to 20c) that create specific motions complementary to a shape of the prongs or barbs 18, 23 operate (together with staged application of traction to the cannula 2) to cause those prongs or barbs 18, 23 to engage with the vessel wall W. Such prong or barb elements 18, 23 can optionally be included in any of the above embodiments where they are not specifically illustrated.

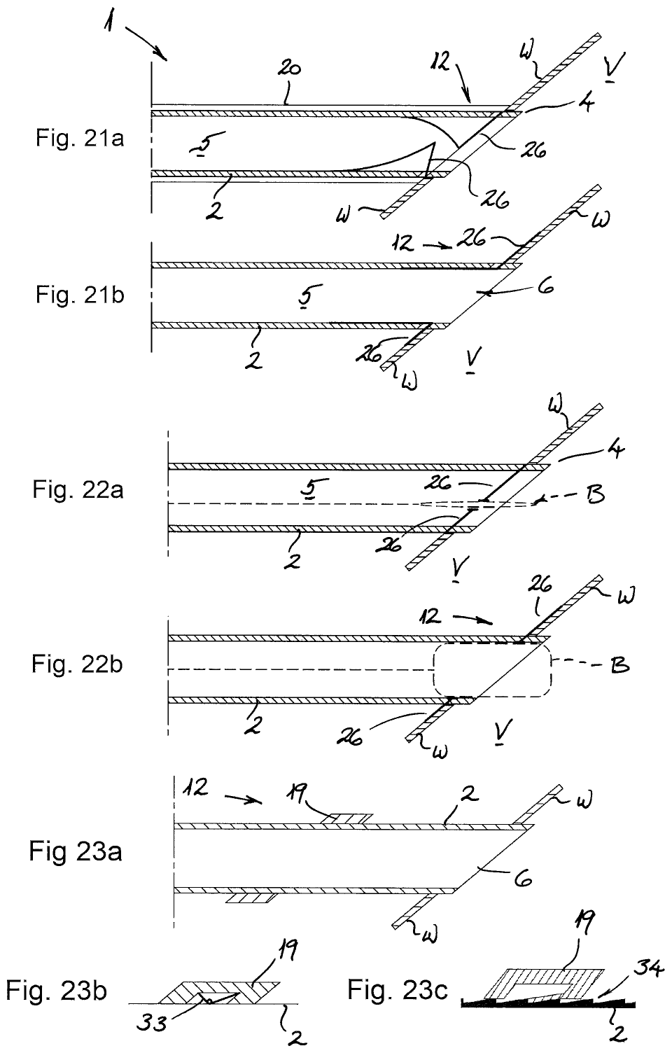

Referring to the embodiments illustrated in FIGS. 21a and 21b and FIGS. 22a and 22b, it will be understood by skilled persons that features of the intravascular part 11 of the fixation mechanism 10 according to many of the embodiments of a vascular access device 1 described above may be applicable to the extravascular part 11 of the fixation mechanism 10 also. In this regard, for example, the embodiment in FIGS. 21a and 21b employs radially outwardly biased cantilevered arm members 26 which extend through apertures or slots 27 formed in the wall of the cannula 2 upon withdrawal of the insertion sheath 20 to form the extravascular part 12 of the fixation mechanism 10, in an analogy to the embodiment of FIGS. 13a to 13c. Further, the embodiment in FIGS. 22a and 22b, for example, employs arm members 26 for deployment via sliding movement through respective apertures or slots, 27 in the sides of the access tube 2 by inflating a vascular balloon B inside the lumen 5 of the access tube 2 to form the extravascular part 12 of the fixation mechanism 10, in an analogy to the embodiment of FIGS. 12a to 12c.

FIG. 23a of the drawings illustrates an embodiment in which the vascular access device 1 has an axially slidable collar 19 as a component of the extravascular part 12 of the fixation mechanism 10, i.e. analogous to the embodiment of FIGS. 8a to 8d above. FIG. 23b shows one variation in which the slidable collar 19 includes a locking means 33 for releasable locking engagement with an outer surface of the cannula 2 upon which the collar 19 is axially movable. FIG. 23c shows a further variation in which the slidable collar 19 includes a ratchet means or latching means 34 adapted for releasable latching engagement with an outer surface of the cannula 2. The slidable collar 19 is adapted to be moved into contact or engagement with an outer surface of the wall W of the blood vessel V.

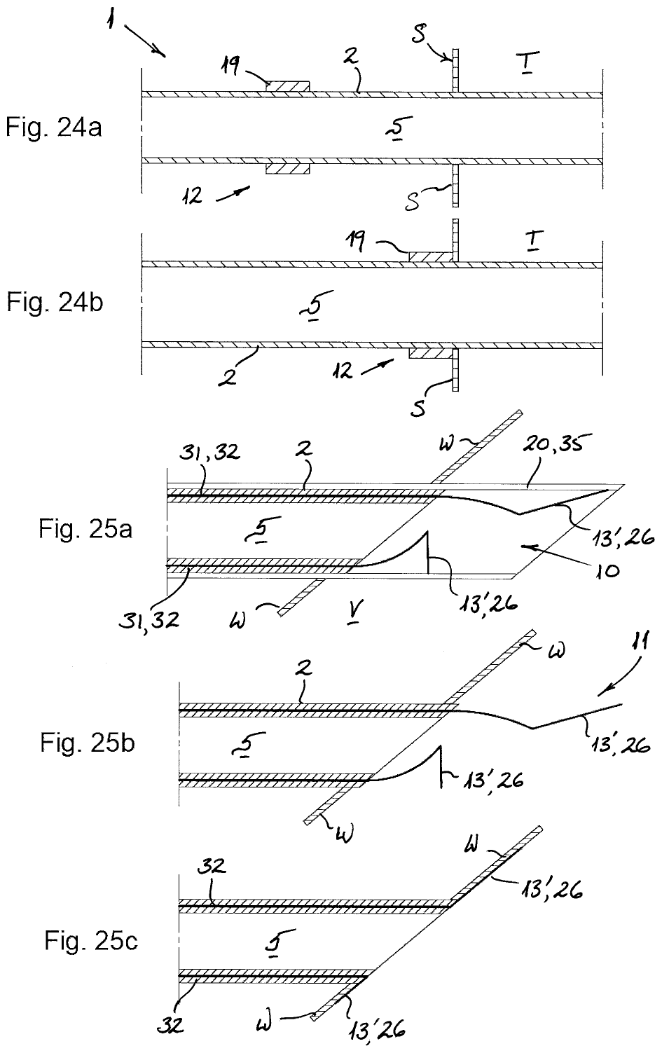

With reference to drawing FIGS. 24a and 24b, a schematic side view is shown of an extravascular part 12 of a fixation mechanism 10 in yet another embodiment of a vascular access device 1 after percutaneous insertion into a blood vessel V. Similar to the embodiment shown in FIGS. 23a to 23c, the vascular access device 1 again includes an axially slidable collar 19 as a component of the extravascular part 12 of the fixation mechanism 10. In this case, however, the slidable collar 19 is adapted to be moved into contact or engagement with the skin S of the patient. That is, while an intravascular part 11 of the fixation mechanism 10 engages with an inner wall of the vessel V to prevent or inhibit withdrawal of the cannular 2, the collar 19 engages with an outer surface of the patient's skin S at the location the cannula 2 emerges percutaneously in order to inhibit or prevent over-insertion of the cannula 2 during use. The collar 19 of this embodiment could again include a locking means 33 or ratchet means or latching means 34 shown in FIG. 23b or 23c for releasable locking or latching engagement with an outer surface of the cannula 2. Alternatively, or in addition, the collar 19 could include a bearing 19′, such as an O-ring, as shown in FIGS. 11a and 11b having a pre-calibrated magnitude of dynamic and static frictional engagement with an outer surface of the cannula 2.

An intravascular part 11 of the fixation mechanism 10 is not shown in FIGS. 24a and 24b, but it could conceivably include elements of the intravascular part 11 of any one of the preceding embodiments. FIGS. 25a to 25c of the drawings illustrate a further example of an intravascular part 11 of a fixation mechanism 10 that could optionally be combined with the extravascular part 12 shown in FIGS. 24a and 24b. This embodiment shares some similarity with the embodiment of FIGS. 18a to 18d. In this case, the intra-vascular part 11 of the fixation mechanism 10 has flange elements 13′ or arm members 26 which, in a non-deployed position shown in FIG. 25a, project forwardly of the distal end region 3 of the cannula 2 from the ends of the slidable actuating member(s) 32. The forwardly projecting flange elements 13′ or arm members 26 are encompassed and/or housed in the non-deployed position by an outer sheath 20 or cover 35. After the tip 4 of the cannula 2 has been inserted through the wall W of the vessel V, the outer sheath 20 or cover 35 is withdrawn to release the flange elements 13′ or arm members 26 for their deployment, as shown in FIG. 25b. The deployment of the flange elements 13′ or arm members 26 involves retracting or withdrawing the actuating member(s) 32 along the channel 31 in the cannula wall. This draws the flange elements 13′ or arm members 26 into engagement with the inner side of the vessel wall W, as shown in FIG. 25c. This action retracts the free length on which the flange elements 13′ or arm members 26 are mounted which not causes the intravascular part 11 to adopt its deployed position, and may also stiffen or lock the intravascular part 11 in its deployed position.

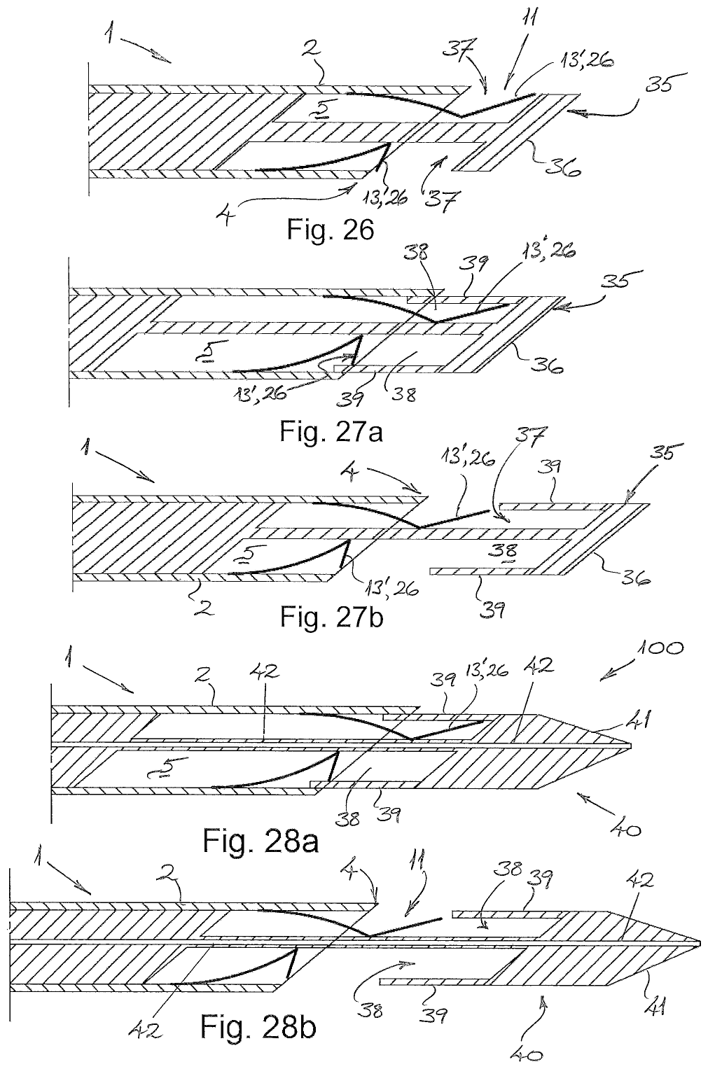

In several of the embodiments of the vascular access device 1, the retracted or collapsed configuration of the fixation mechanism 10 includes members or elements 13, 22, 26 that project forwards from the distal end region 3 of the cannula 2 of the device. In such cases, there is a conceivable risk of trauma to an opposite wall W of the blood vessel V from contact of these projecting structures during insertion of the device 1 into the vessel V. Equally, this contact could result in damage to the fixation mechanism 10 itself. Similarly, contact between such projecting members or elements 13, 22, 26 and a haemostasis valve of a tear-away sheath used to facilitate percutaneous insertion of the vascular access device 1 into the vessel V may occur. In such a case, mutual damage to the haemostasis valve and the fixation mechanism 10 could result. There is further a possibility of projecting members or elements 13′, 22, 26 of the fixation mechanism 10 interacting with the edges of the breach in the vessel wall W and/or with extravascular tissue T during removal or explantation. To this end, and with reference to FIG. 26 of the drawings, the vascular access device 1 may therefore optionally include a protector or guard member 35 which provides a blunt, atraumatic end-stop. More particularly, that protector or guard member 35 will typically have atraumatic geometries (e.g. smooth surfaces and no sharp corners, edges, or burrs) at locations that may make contact or interact with the blood vessel wall W or haemostasis valve. Thus, the protector or guard member 35 may have a flat distal end 36 (as shown) or a rounded distal end and may optionally be formed from a relatively soft, flexible material designed to avoid inflicting any trauma on tissue of the vessel V. Furthermore, the protector or guard member 35 is configured to prevent any of the projecting members or elements 13′, 22, 26 of the fixation mechanism 10 from making contact with an opposite wall W of the vessel V, or with the haemostasis valve of a tear-away introducer sheath. To this end, the guard member 35 may define a recess 37 adjacent the distal end 36 to accommodate the projecting members or elements 13′, 22, 26. The guard member 35 is introduced through the lumen 5 of the cannula 2 and is designed for use during both insertion or implantation, as well as during explantation or removal, of the vascular access device 1.

With reference to the embodiment in drawing FIGS. 27a and 27b, the recess 37 of the protector or guard member 35 may comprise a chamber 38 with side walls 39. In this way, the chamber 38 of the guard member 35 is configured to accommodate and also substantially encompass or enclose projecting members or elements 13′, 22, 26 of the fixation mechanism 10. Thus, the chamber 38 and side walls 39 hide or obscure the projecting members or elements 13′, 22, 26 of fixation mechanism 10 in their collapsed or non-deployed configuration and isolate them from contact with either the opposite vessel wall W or the haemostasis valve of the tear-away sheath (not shown). When the device 1 is being inserted into a target vessel V, the protector/guard member 35 may need to be advanced further through the lumen of the cannula, as seen in FIG. 27b, in order to free the projecting members or elements 13′, 22, 26 of the fixation mechanism 10 from the recesses or spaces 36 in the guard member 35 and thereby allow their deployment. For variations in which the intravascular part 11 of fixation mechanism 10 is outwardly biased and deploys by elastic recovery (e.g. after retraction of a confining sheath 20), a protector or guard member 35 with a cavity 38 that encloses the fixation mechanism 10 could facilitate a similar operation. In that case, as the protector or guard member 35 is advanced, this action releases the intravascular part 11 of the fixation mechanism 10 from the cavity (see FIG. 27b) and allows it to deploy via elastic recovery.

After the fixation mechanism 10 has been activated, the intra-luminal space 3 within the cannula 2 should be free to allow removal (i.e. withdrawal) of the protector or guard member 35. Conversely, when the device 1 is being explanted from the blood vessel V, the protector or guard member 35 should then be correctly positioned (axially) within the vascular access device 1 such that projecting structures 13′, 22, 26 of the fixation mechanism 10 may again be retracted into the chamber 38 into the protector or guard member 35 when the fixation mechanism 10 is deactivated. That is, the protector or guard member 35 may be reintroduced into the lumen 5 of the cannula 2 prior to the device 1 being removed or explanted from the vessel V in order to again accommodate or house the member(s) or part(s) 13′, 22, 26 of the fixation mechanism 10 when the deployment of the fixation mechanism 10 is ended. In this way, the protector or guard member 35 may also operate to protect the tissues of the patient from such projecting structures 13′, 22, 26 during the removal or withdrawal of the vascular access device 1 from the patient. It is noted that the protector or guard member 35 may optionally also be in the form of a short outer sheath or cover that encompasses or covers both the cannula 2 and any projecting member or element 13, 22, 26 of the fixation mechanism 10 in the collapsed/non-activated configuration. The sheath-like cover or guard member 35 in this embodiment may be positioned over the fixation mechanism 10 to cover it during the process of inserting the access device 1 through a haemostasis valve of the introducer sheath. After the distal end region 3 of the device 1 is inside the delivery sheath, the temporary covering may be retracted. The sheath-like guard member 35 may have a blunt, atraumatic geometry and be comprised of a relatively soft, flexible material.

Finally, with reference to FIGS. 28a and 28b of the drawings, an embodiment of a vascular access system 100 is illustrated comprising a vascular access device 1 as just described and a dilator 40 for gradually expanding or widening a breach or opening formed in a wall W of a blood vessel V. To this end, the dilator 40 has a conically tapered distal end 41 which engages with edges of the breach in the vessel wall W and gradually and atraumatically expands or dilates that breach as the tapered end 41 of the dilator 40 is advanced into the blood vessel V. In this way, the dilator 40 cooperates with the access tube 2 of the vascular access device 1 to guide and introduce the tip 4 through the breach or opening in the wall W of the vessel V. To assist the process, the dilator 40 has a central channel 42 for accommodating a guidewire (not shown) to guide a path of the dilator 40, and thereby of the tip 4 of the vascular access tube 2, through the breach in the wall W of the vessel V. It will be noted from the similarity to FIGS. 27a and 27b that the dilator 40 incorporates some of the features of the protector or guard member 35. In particular, the dilator 40 defines a chamber 38 surrounded by side walls 39 and configured to house or accommodate the projecting elements 13′, 22, 26 of the intravascular part 11 of the fixation mechanism 10. In this way, the dilator 40 is designed to keep these elements from any unwanted interaction with an opposite wall of the vessel during insertion. The dilator 40 also provides for geometrically smooth (atraumatic) transitions between the guidewire (not shown) and the conical end 41, and between the side walls 39 and the cannula 2, with no sharp edges, corners, burrs. Once the distal tip 4 of the cannula 2 has passed through the breach into the vessel V, further advancing the dilator 40 along the guidewire into lumen L away from the cannula 2, as represented in FIG. 28b, acts to release the intravascular part 11 of the fixation mechanism 10 from the chamber 39 so that the projecting elements 13′, 26 can then be deployed. After their deployment, the dilator 40 (and optionally the guidewire) may then be withdrawn from the blood vessel V through the lumen 5 of the cannula 2.

Although specific embodiments of the invention are illustrated and described herein, it will be appreciated by persons of ordinary skill in the art that a variety of alternative and/or equivalent implementations exist. It should be appreciated that each exemplary embodiment is an example only and is not intended to limit the scope, applicability, or configuration in any way. Rather, the foregoing summary and detailed description will provide those persons skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that the terms “comprise”, “comprising”, “include”, “including”, “contain”, “containing”, “have”, “having”, and any variations thereof, used in this document are intended to be understood in an inclusive (i.e. non-exclusive) sense, such that the process, method, device, apparatus, or system described herein is not limited to the features, integers, parts, elements, or steps recited but may include other features, integers, parts, elements, or steps not expressly listed and/or inherent to such process, method, device, apparatus, or system. Furthermore, the terms “a” and “an” used herein are intended to be understood as meaning one or more unless explicitly stated otherwise. In addition, reference to positional terms, such as “lower” and “upper”, used in the above description are to be taken in context of the embodiments depicted in the figures, and are not to be taken as limiting the invention to the literal interpretation of the term but rather as would be understood by the skilled addressee in the appropriate context.

The terms “proximal” and “distal” are used to refer to the opposite ends of a medical device, such as the vascular access devices disclosed herein. As used herein, the “proximal” end region of the device is the end region near a practitioner during use, while the distal end region is the region at the opposite end of the device remote from the practitioner during use. For example, the proximal end region of a vascular access device is the end closest to the practitioner during the insertion or deployment of the vascular access device. The distal end is the end opposite the proximal end along the longitudinal direction of the medical anchor device. In this case, the tip of the vascular access tube at the distal end region is designed for insertion into the blood vessel.

1. A vascular access device, comprising:
a vascular access tube having a distal end region terminating in a tip to be inserted into a blood vessel of a patient and at least one lumen for infusing a medicament and/or for introducing one or more catheters there-through into the blood vessel; and
a fixation mechanism operable to secure or fix the tip of the vascular access tube within the blood vessel, wherein the fixation mechanism operates to secure or fix the tip at or adjacent a wall of the blood vessel at a point of insertion of the tip into the blood vessel.

2. A device according to claim 1, wherein the tip has an opening to provide fluid communication between the at least one lumen of the vascular access tube and the blood vessel, wherein the fixation mechanism operates to position and to secure or fix the opening at or adjacent the wall of the blood vessel at the point of insertion of the tip.

3. A device according to claim 1 or claim 2, wherein the fixation mechanism is configured to inhibit or prevent withdrawal of the tip from the blood vessel and/or to inhibit or prevent over-insertion of the tip into the blood vessel.

4. A vascular access device, comprising:
a vascular access tube having a distal end region terminating in a tip for insertion into a blood vessel of a patient and at least one lumen for infusing a medicament and/or for introducing one or more catheters there-through into the blood vessel; and
a fixation mechanism operable to secure or fix the distal end region of the vascular access tube with respect to the patient to inhibit or prevent withdrawal of the tip from the blood vessel and/or over-insertion of the tip into the blood vessel.

5. A device according to claim 4, wherein the tip of the vascular access tube has an opening for communication between the at least one lumen of the access tube and the blood vessel of the patient into which the tip is inserted, wherein the fixation mechanism is configured to position and fix the opening of the tip at or adjacent a wall of the blood vessel at a point of insertion of the tip.

6. A device according to any one of claims 1 to 5, wherein the fixation mechanism is configured to secure or fix the vascular access tube such that the access tube extends longitudinally at a predetermined angle with respect to the blood vessel at a point of insertion of the tip.

7. A device according to claim 6, wherein the predetermined angle is within the range of about 20 degrees to about 70 degrees.

8. A device according to any one of claims 1 to 7, wherein the fixation mechanism comprises an intravascular part configured to engage with an inner surface of the wall of the blood vessel through which the tip is inserted to inhibit or prevent withdrawal of the tip of the vascular access tube from the blood vessel.

9. A device according to claim 8, wherein the intravascular part of the fixation mechanism includes at least one member that is movable between a retracted or non-deployed position for insertion of the tip of the access tube into the blood vessel and an extended or deployed position for engaging the inner surface of the wall of the blood vessel to inhibit or prevent withdrawal of the tip.

10. A device according to claim 9, wherein the at least one member of the intra-vascular part projects laterally or radially outwardly from the vascular access tube in its extended or deployed position.

11. A device according to claim 9 or claim 10, wherein the at least one member of the intravascular part comprises a barb or prong configured for piercing the wall of the blood vessel.

12. A device according to any of claims 1 to 11, wherein the fixation mechanism comprises an extravascular part configured to engage with tissue of the patient outside of the blood vessel.

13. A device according to claim 12, wherein the extravascular part is configured to engage with an outer surface of the wall of the blood vessel through which the tip is inserted or with tissue that adjoins or encases the wall of the blood vessel, with the extravascular part preferably cooperating with the intra-vascular part to capture or clamp the wall of the blood vessel there-between.

14. A device according to claim 12 or claim 13, wherein the extravascular part of the fixation mechanism includes at least one member that is movable between a non-deployed position for insertion of the tip of the access tube into the blood vessel and a deployed position to inhibit or prevent over-insertion of the tip.

15. A device according to claim 14, wherein the at least one member of the extra-vascular part includes at least one barb or prong for engaging in subcutaneous tissue of the patient in its deployed position.

16. A device according to any of claims 1 to 15, wherein the fixation mechanism comprises an activation member operably associated or connected with the distal end region of the vascular access tube, wherein the activation member is configured for operation by a user at a proximal end region of the access tube to activate or operate the fixation mechanism during or upon insertion of the tip of the access tube into the blood vessel.

17. A device according to claim 16, wherein the activation member comprises a sheath, such as a delivery sheath, that covers or accommodates the distal end region of the vascular access tube during insertion of the tip into the blood vessel, wherein the sheath is configured to be withdrawn from the distal end region, and wherein withdrawal of the sheath operates to cause the at least one member of the intravascular part and/or the at least one member of the extra-vascular part of the fixation mechanism to move from its respective retracted or non-deployed position to its extended or deployed position.

18. A device according to claim 16, wherein the activation member is configured for axially slidable movement, preferably as a sleeve or cannula element, within the lumen of the vascular access tube, wherein the activation member is slidably movable towards the distal end region to cause the at least one member of the intravascular part and/or the at least one member of the extravascular part of the fixation mechanism to move from its respective retracted or non-deployed position to its extended or deployed position.

19. A device according to any one of claims 1 to 18, comprising a protector or guard member at or adjacent the tip of the access tube with a recess to accommodate a member or part of the fixation mechanism, and especially of the intravascular part of the fixation mechanism, to prevent it from inadvertently making contact or interacting with the blood vessel during the insertion of the access tube into the blood vessel.

20. A device according to claim 19, wherein the protector or guard member includes a chamber configured to accommodate, encompass, or substantially house the said member or part of the fixation mechanism.

21. A vascular access system comprising:
a vascular access device according to any of the preceding claims, and
a dilator for gradually widening a breach or an opening formed in a wall of a blood vessel,
wherein the dilator is adapted to cooperate with the access tube of the vascular access device to guide and/or introduce the tip of the distal end region of the access tube through the breach or opening in the wall of the vessel.

22. A vascular access system according to claim 21, wherein the tip of the access tube is configured to provide a smooth or gradual, preferably tapered, transition to an outer periphery of the dilator.

23. A vascular access system according to claim 21 or claim 22, wherein the dilator is sized and/or adapted to be withdrawn or removed from the patient through the lumen of the access tube.

24. A vascular access system according to any one of claims 21 to 23, wherein the dilator includes a channel, preferably centrally or axially, for accommodating a guidewire to guide a path of the dilator and thereby of the tip of the distal end region of the vascular access tube through the breach or opening formed in the wall of the vessel.

25. A vascular access system according to any one of claims 21 to 24, wherein the dilator includes a cavity or chamber configured to house or accommodate one or more members or parts of the fixation mechanism of the access device.

26. A method of implanting a vascular access device in a patient, the method comprising steps of:
inserting a vascular access tube into a patient, the access tube having a tip for introduction through a wall of a blood vessel of the patient and at least one lumen for introducing one or more catheters there-through into the blood vessel; and
activating a fixation mechanism provided at a distal end region of the vascular access tube to secure or fix the distal end region of the access tube with respect to the patient, whereby the fixation mechanism secures or fixes the tip of the access tube at or adjacent the wall of the blood vessel at a point of entry through the wall.

27. A method according to claim 26, wherein the step of inserting the access tube into the patient, preferably percutaneously, comprises introducing the tip of the access tube through the wall of the blood vessel over a guidewire, preferably with the aid of a dilator.

28. A method according to claim 26, wherein the step of inserting the access tube into the patient, preferably percutaneously, comprises introducing the vascular access tube via a previously inserted delivery sheath, preferably percutaneously inserted, at the intended site of implantation.

29. A method according to any of claims 26 to 28, wherein the step of activating the fixation mechanism comprises moving at least one member of an intravascular part of the fixation mechanism to an extended or deployed position to engage with an inner surface of the wall of the blood vessel through which the tip was inserted.

30. A method according to any of claims 26 to 29, wherein the step of activating the fixation mechanism comprises moving at least one member of an extravascular part of the fixation mechanism to a deployed position to engage with an outer side or surface of the wall of the blood vessel, or with tissue that adjoins or encases the wall of the blood vessel, through which the tip has been inserted.

31. A method according to any of claims 26 to 30, wherein the step of activating the fixation mechanism comprises moving at least one member of an extravascular part of the fixation mechanism to a deployed position to engage subcutaneous tissue of the patient.

32. A method according to any of claims 26 to 31, comprising a step of releasing one or more members or parts of the fixation mechanism from a protector or guard member at a tip of the vascular access tube after inserting the vascular access tube into a patient and before or while activating the fixation mechanism.

33. A method of percutaneously explanting a vascular access device from a patient, the vascular access device having a vascular access tube with a tip inserted through a wall of a blood vessel of the patient and defining at least one lumen for introducing one or more catheters there-through into the blood vessel, the method comprising:
deactivating a fixation mechanism at a distal end region of the vascular access tube to release the distal end region of the access tube with respect to the patient; and
removing the vascular access tube from the patient, e.g. percutaneously.

34. A method according to claim 33, wherein the step of deactivating the fixation mechanism comprises moving at least one member of an intravascular part and/or an extravascular part of the fixation mechanism from an extended or deployed position to a retracted or non-deployed position to release the distal end region of the access tube.

35. A method according to claim 33 or claim 34, comprising a step of housing or accommodating one or more members or parts of the fixation mechanism in a protector or guard member at a tip of the vascular access tube after the step of deactivating the fixation mechanism and before removing the vascular access tube from the patient.
